(19) United States
(12) Patent Application Publication
(10) Pub. No.: US 2006/0178351 A1
(43) Pub. Date: Aug. 10, 2006

(54) Treatment of immune-mediated disorders with active vitamin D compounds alone or in combination with other therapeutic agents

(75) Inventors: John G. Curd, Hillsborough, CA (US); Martha J. Whitehouse, San Francisco, CA (US); Jeffrey L. Cleland, San Carlos, CA (US)

Correspondence Address: STERNE, KESSLER, GOLDSTEIN & FOX PLLC, 1100 NEW YORK AVENUE, N.W., WASHINGTON, DC, 20005, US

(73) Assignee: Novacea, Inc.

(21) Appl. No.: 11/298,989

(22) Filed: Dec. 12, 2005

Related U.S. Application Data
(63) Continuation-in-part of application No. PCT/US04/18184, filed on Jun. 10, 2004.
(60) Provisional application No. 60/477,398, filed on Jun. 11, 2003.
(60) Provisional application No. 60/569,243, filed on May 10, 2004.

(51) Int. Cl. A61K 31/59

(52) U.S. Cl. 514/167

(57) ABSTRACT

The present invention relates to a method for treating, ameliorating, or preventing immune-mediated disorders in an animal by administering to the animal active vitamin D compounds or mimics thereof The invention further relates to a method for treating, ameliorating, or preventing immune-mediated disorders in an animal by administering to the animal active vitamin D compounds or mimics thereof in combination with other therapeutic agents.

### BACKGROUND OF THE INVENTION

1. Field of the Invention

The present invention relates to a method for treating or ameliorating immune-mediated disorders in an animal by administering to the animal active vitamin D compounds or mimics thereof. The invention further relates to a method for treating or ameliorating immune-mediated disorders in an animal by administering to the animal active vitamin D compounds or mimics thereof in combination with other therapeutic agents.

2. Related Art

Vitamin D is a fat soluble vitamin which is essential as a positive regulator of calcium homeostasis. (See Harrison's Principles of Internal Medicine: Part Thirteen, “Disorders of Bone and Mineral Metabolism,” Chapter 353, pp. 2214-2226, A. S. Fauci et al., (eds.), McGraw-Hill, New York (1998)). The active form of vitamin D is 1α,25-dihydroxyvitamin D₃, also known as calcitriol. Specific nuclear receptors for active vitamin D compounds have been discovered in cells from diverse organs not involved in calcium homeostasis. (Miller et al., Cancer Res. 52:515-520 (1992)). In addition to influencing calcium homeostasis, active vitamin D compounds have been implicated in osteogenesis, modulation of immune response, modulation of the process of insulin secretion by the pancreatic B cell, muscle cell function, and the differentiation and growth of epidermal and hematopoietic tissues.

Moreover, there have been many reports demonstrating the utility of active vitamin D compounds in the treatment of hyperproliferative diseases, (e.g., cancer, psoriasis). For example, it has been shown that certain vitamin D compounds and analogs possess potent antileukemic activity by virtue of inducing the differentiation of malignant cells (specifically, leukemic cells) to non-malignant macrophages (monocytes) and are useful in the treatment of leukemia. (Suda et al., U.S. Pat. No. 4,391,802; Partridge et al., U.S. Pat. No. 4,594,340). Anti-proliferative and differentiating actions of calcitriol and other vitamin D₃ analogues have also been reported with respect to the treatment of prostate cancer. (Bishop et al., U.S. Pat. No. 5,795,882). Active vitamin D compounds have also been implicated in the treatment of skin cancer (Chida et al., Cancer Research 45:5426-5430 (1985)), colon cancer (Disman et al., Cancer Research 47:21-25 (1987)), and lung cancer (Sato et al., Tohoku J. Exp. Med. 138:445-446 (1982)). Other reports suggesting important therapeutic uses of active vitamin D compounds are summarized in Rodriguez et al., U.S. Pat. No. 6,034,079.

Active vitamin D compounds have also been administered in combination with other pharmaceutical agents, in particular cytotoxic agents for the treatment of hyperproliferative disease. For example, it has been shown that pretreatment of hyperproliferative cells with active vitamin D compounds followed by treatment with cytotoxic agents enhances the efficacy of the cytotoxic agents (U.S. Pat. No. 6,087,350; WO 01/64251).

Vitamin D is involved in normal cell growth and maturation. Its role as an immune modulator has been gaining more attention. Several immune-mediated disorders, e.g., multiple sclerosis, Sjögren's Syndrome, rheumatoid arthritis, thyroiditis and Crohn's disease, have been linked with low vitamin D level. For review, see Deluca et al., FASEB J. 15:2579-2585 (2001); Long et al., Pediat. Infect. Dis. J. 18:283-290 (1999); Cantorna, Proc. Soc. Exp. Biol. Med. 223:230-233 (2000)).

Although the administration of active vitamin D compounds may result in substantial therapeutic benefits, the treatment of hyperproliferative diseases, immune-mediated disorders and other diseases with such compounds is limited by the effects these compounds have on calcium metabolism. At the levels required in vivo for effective use as anti-proliferative/immunosuppressive agents, active vitamin D compounds can induce markedly elevated and potentially dangerous blood calcium levels by virtue of their inherent calcemic activity. That is, the clinical use of calcitriol and other active vitamin D compounds as anti-proliferative agents is severely limited by the risk of hypercalcemia.

A great deal of research has gone into the identification of vitamin D analogs and derivatives that maintain an anti-proliferative effect but have a decreased effect on calcium metabolism. Hundreds of compounds have been created, many with reduced hypercalcemic effects, but no compounds have been discovered that maintain anti-proliferative activity while completely eliminating the hypercalcemic effect.

It has been shown that the problem of systemic hypercalcemia can be overcome by “pulsed-dose” administration of a sufficient dose of an active vitamin D compound such that an anti-proliferative effect is observed while avoiding the development of severe hypercalcemia. According to U.S. Pat. No. 6,521,608, the active vitamin D compound may be administered no more than every three days, for example, once a week at a dose of at least 0.12 μg/kg per day (8.4 μg in a 70 kg person). Pharmaceutical. compositions used in the pulsed-dose regimen of U.S. Pat. No. 6,521,608 comprise 5-100 μg of active vitamin D compound and may be administered in the form for oral, intravenous, intramuscular, topical, transdermal, sublingual, intranasal, intratumoral or other preparations.

Autoimmune Diseases

Autoimmune diseases are caused when the body's immune system, which is meant to defend the body against bacteria, viruses, and any other foreign product, malfunctions and produces antibodies against healthy tissue, cells and organs. Antibodies, T cells and macrophages provide beneficial protection, but can also produce harmful or deadly immunological responses.

The principle mechanisms by which auto-antibodies can produce an autoimmune disease are complement-dependent lytic destruction of the target cell, opsonization, formation of immune complexes, blockade of receptor sites for physiological ligands, and stimulation of cell surface receptors. The auto-antibody can bind to cell surface receptors and either inhibit or stimulate the specialized function of the cell (Paul, W. E., ed., Fundamental Immunology, Raven Press, New York, Chapter 31, p. 839 (1989)).

Autoimmune diseases can be organ specific or systemic and are provoked by different pathogenic mechanisms. Organ specific autoimmunization is characterized by tolerance and suppression within the T cell compartment, aberrant expression of major-histocompatibility complex (MHC) antigens, antigenic mimicry and allelic variations in MHC genes. Systemic autoimmune diseases involve polyclonal B cell activation and abnormalities of immunoregulatory T cells, T cell receptors and MHC genes. Examples of organ specific autoimmune diseases include diabetes, hyperthyroidism, autoimmune adrenal insufficiency, pure red cell anemia, multiple sclerosis and rheumatic carditis. Representative systemic autoimmune diseases include systemic lupus erythematosus, rheumatoid arthritis, chronic inflammation, Sjögren's syndrome, polymyositis, dermatomyositis and scleroderma.

Current treatment of autoimmune diseases involves administering immunosuppressive agents such as cortisone, methotrexate, azathioprine, and cyclophosphamide or combinations thereof. The dilemma faced when administering immunosuppressive agents, however, is the more effectively the autoimmune disease is treated, the more defenseless the patient is left to attack from infections. Accordingly, there is a need for improved, safer treatments that have long-lasting effects for the prevention and treatment of autoimmune disorders. In particular, there is a need for treatments that are more specific and less toxic than the currently available therapeutic agents.

Inflammatory Disorders

Inflammation plays a fundamental role in host defenses and the progression of immune-mediated diseases. The inflammatory response is initiated in response to injury (e.g., trauma, ischemia, and foreign particles) and infection (e.g., bacterial or viral infection) by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events result in swelling, redness, warmth (altered heat patterns), and pus formation at the site of injury or infection.

Cytokines and prostaglandins control the inflammatory response, and are released in an ordered and self-limiting cascade into the blood or affected tissues. This release of cytokines and prostaglandins increases the blood flow to the area of injury or infection, and may result in redness and warmth. Some of these chemicals cause a leak of fluid into the tissues, resulting in swelling. This protective process may stimulate nerves and cause pain. These changes, when occurring for a limited period in the relevant area, work to the benefit of the body.

A delicate well-balanced interplay between the humoral and cellular immune elements in the inflammatory response enables the elimination of harmful agents and the initiation of the repair of damaged tissue. When this delicately balanced interplay is disrupted, the inflammatory response may result in considerable damage to normal tissue and may be more harmful than the original insult that initiated the reaction. In these cases of uncontrolled inflammatory responses, clinical intervention is needed to prevent tissue damage and organ dysfunction. Diseases such as rheumatoid arthritis, osteoarthritis, Crohn's disease, psoriasis, and inflammatory bowel disease are characterized by chronic inflammation.

Current treatments for inflammatory disorders involve symptomatic medications and immunosuppressive agents to control symptoms. For example, nonsteroidal anti-inflammatory drugs (NSAIDs) such as aspirin, ibuprofen, fenoprofen, naproxen, tolmetin, sulindac, meclofenamate sodium, piroxicam, flurbiprofen, diclofenac, oxaprozin, nabumetone, etodolac, and ketoprofen have analgesic and anti-inflammatory effects. However, NSAIDs are believed not to be capable of altering progression of the disease. (Tierney et al., eds, Current Medical Diagnosis & Treatment, 37 ed., Appleton & Lange (1998), p. 793). Moreover, NSAIDs frequently cause gastrointestinal side effects, affect the lower intestinal tract causing perforation or aggravating inflammatory bowel disease, produce renal toxicity and prolong bleeding time. Corticosteroids are another class of drugs that are commonly used to control inflammatory symptoms. Corticosteroids, like NSAIDs, do not alter the natural progression of the disease, and thus, clinical manifestations of active disease commonly reappear when the drug is discontinued. The serious problem of untoward reactions resulting from prolonged corticosteroid therapy (e.g., osteoporosis, increased risk of infection, increased appetite, hypertension, edema, peptic ulcers, psychoses) greatly limits its long-term use.

Low doses of immunosuppressive agents such as cytotoxic agents are also commonly used in treatment of inflammatory disorders. For example, methotrexate, an antagonist of folic acid, is often used in treatment of psoriasis, rheumatoid arthritis and other inflammatory diseases. Methotrexate, like other cytotoxic agents, frequently causes stomatitis, erythema, alopecia, nausea, vomiting, diarrhea, and damage to major organs such as kidney and liver. The long-term usage of immunosuppressive agents usually leaves the patient defenseless to infections.

New treatments for inflammatory disorders are constantly being sought. In particular, any new treatment that targets the underlying cause of an inflammatory disease, reduces the dosage and/or frequency of administration of agents currently being used, or is capable of making a currently used treatment more effective is constantly being sought.

Transplant Rejection

Transplant rejection occurs in individuals receiving tissue from genetically non-identical individuals and is mediated by T cell-dependent mechanisms. To prevent allograft rejection, immunosuppressive agents such as calcineurin phosphatase inhibitors (e.g., cyclosporin A, FK506, and rapamycin) and glucocorticoids which directly or indirectly interfere with interleukin (IL)-2 signaling are administered to transplant recipients (see, e.g., Borel, Pharmacol. Rev. 42:260-372 (1989); Morris, P. J., Curr. Opin. Immunol. 3:748-751 (1991); Sigal et al., Ann. Rev. Immunol. 10:519-560 (1992); and L'Azou et al., Arch. Toxicol. 73:337-345 (1999)). The effect of immunosuppressive agents is short-lasting, and thus, transplant recipients normally require life-long treatment of immunosuppressive agents to prevent transplant rejection. As a result of the long-term administration of immunsuppressive agents, transplant recipients suffer from serious adverse effects such as, e.g., the development of infections and tumors.

Cyclosporin A, FK506, and rapamycin are among the most commonly used immunosuppressive agents today. These immunosuppressive agents act indiscriminately on all T cells by impairing T cell receptor (TCR) signal transduction. The long-term administration of cyclosporin A or FK506 to transplant recipients results in numerous serious adverse effects including, but not limited to, changes in renal tubules, tremor, hirsutism, hypertension, hyperlipidemia, gum hyperplasia, neurotoxicity, gastrointestinal complications, hyperkalemia, hyperglycemia, and diabetes. See e.g., Hardman et al., eds., Goodman & Gilman's The Pharmacological Basis Of Therapeutics, 10th Ed, Mc-Graw-Hill, New York (2001), pp. 1468-1470. An alternative to the administration of immunosuppressive agents such as cyclosporin A or FK506 to prevent allograft rejection is the administration of agents that modulate TCR activation, the proliferation of T helper (Th)1/Th2 cells and/or the differentiation of Th1/Th2 cells. Examples of such agents include, but are not limited to, CTLA-41g, anti-CD40 antibodies, anti-CD40 ligand antibodies, anti-IL-2 receptor antibodies, and anti-CD28 antibodies. Although these agents are more target specific, anaphylactic reactions can and do occur following their administration to an individual. Further, lymphoproliferative and opportunistic infections are common adverse side effects associated with the administration of such agents.

Accordingly, there is a need for improved, safer treatments that have long-lasting effects for the prevention and treatment of transplant rejection. In particular, there is a need for treatments that are more specific and less toxic than the currently available therapeutic agents.

### SUMMARY OF THE INVENTION

One aspect of the present invention is a method for treating, ameliorating, or preventing an immune-mediated disorder in an animal comprising administering to the animal an active vitamin D compound or a mimic thereof. In a second preferred aspect of the invention the active vitamin D compound or a mimic thereof has a reduced hypercalcemic effect, allowing higher doses of the compound to be administered to an animal without inducing hypercalcemia. In another embodiment of the invention the active vitamin D compound or a mimic thereof is administered in a pulsed-dose fashion so that high doses of the active vitamin D compound or a mimic thereof can be administered to an animal without inducing hypercalcemia. Another aspect of the present invention is a method for treating, ameliorating, preventing an immune-mediated disorder in an animal comprising administering to the animal an active vitamin D compound or a mimic thereof in combination with one or more therapeutic agents.

In preferred embodiments of the invention, the immune-mediated disorder is an autoimmune disorder, an inflammatory disorder or transplant rejection. In preferred embodiments, the one or more therapeutic agents are selected from an immunomodulatory agent, an anti-angiogenic agent, an anti-inflammatory agent, or a dermatological agent. In further embodiments, a combination of therapeutic agents is administered. In one embodiment of the invention, vitamin D administration can start prior to administration of the one or more therapeutic agents and/or continue during and beyond administration of the one or more therapeutic agents. In another embodiment of the invention, the method of administering an active vitamin D compound in combination with one or more therapeutic agents is repeated more than once.

The combination of an active vitamin D compound or a mimic thereof with one or more therapeutic agents of the present invention can have additive potency or an additive therapeutic effect. The invention also encompasses synergistic combinations where the therapeutic efficacy is greater than additive. Preferably, such combinations also reduce or avoid unwanted or adverse effects. In certain embodiments, the combination therapies encompassed by the invention provide an improved overall therapy relative to administration of an active vitamin D compound or a mimic thereof or any therapeutic agent alone. In certain embodiments, doses of existing or experimental therapeutic agents can be reduced or administered less frequently which increases patient compliance, thereby improving therapy and reducing unwanted or adverse effects.

Further, the methods of the invention are useful not only with previously untreated patients but also useful in the treatment of patients partially or completely refractory to current standard and/or experimental therapies for immune-mediated disorders. In a preferred embodiment, the invention provides therapeutic methods for the treatment or amelioration of immune-mediated disorders that have been shown to be or may be refractory or non-responsive to other therapies.

### DETAILED DESCRIPTION OF THE INVENTION

One aspect of the present invention is a method for treating, ameliorating, or preventing an immune-mediated disorder in an animal comprising administering to the animal an active vitamin D compound or a mimic thereof. In a second preferred aspect of the invention the active vitamin D compound or a mimic thereof has a reduced hypercalcemic effect, allowing higher doses of the compound to be administered to an animal without inducing hypercalcemia. A further aspect of the present invention is a method for treating or ameliorating immune-mediated disorders in an animal comprising administering to the animal an active vitamin D compound or a mimic thereof in a pulsed-dose fashion so that high doses of the active vitamin D compound or a mimic thereof can be administered to an animal without inducing hypercalcemia.

Another aspect of the present invention is a method for treating, ameliorating, or preventing immune-mediated disorders in an animal comprising administering to the animal an active vitamin D compound or a mimic thereof in combination with one or more therapeutic agents, which therapeutic agents are currently being used, have been used, or are known to be useful in the treatment, amelioration, or prevention of an immune-mediated disorder.

The methods described herein are useful for the treatment or amelioration of autoimmune disorders including, but not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behçet's disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barré syndrome, Hashimoto's thyroiditis, idiopathic pulmonary fibrosis, idiopathic thrombocytopenia purpura, IgA neuropathy, juvenile arthritis, lichen planus, Ménière's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, rheumatoid arthritis, sarcoidosis, scleroderma, progressive systemic sclerosis, Sjögren's syndrome, Goodpasture's syndrome, stiff-man syndrome, systemic lupus erythematosus, lupus erythematosus, Takayasu's arteritis, temporal arteritis, giant cell arteritis, ulcerative colitis, uveitis, vasculitides such as dermatitis herpetiformis vasculitis, vitiligo, and Wegener's granulomatosis. The methods described herein are particularly useful for the treatment or amelioration of autoimmune disorders characterized by increased T cell infiltration of lymphocytes into affected dermal or epidermal tissues, autoimmune disorders characterized by increased T cell activation and/or abnormal antigen presentation, or autoimmune disorders characterized by increased B cell activation and/or abnormal antibody production.

The methods described herein are useful for the treatment or amelioration of inflammatory disorders including, but not limited to, asthma, encephalitis, inflammatory bowel disease (e.g., Crohn's disease and ulcerative colitis), chronic obstructive pulmonary disease, inflammatory osteolysis, allergic disorders, septic shock, pulmonary fibrosis (e.g., idiopathic pulmonary fibrosis), inflammatory vasculitides (e.g., polyarteritis nodosa, Wegener's granulomatosis, Takayasu's arteritis, temporal arteritis, and lymphomatoid granulomatosus), post-traumatic vascular angioplasty (e.g., restenosis after angioplasty), undifferentiated spondyloarthropathy, undifferentiated arthropathy, arthritis, inflammatory osteolysis, chronic hepatitis, and chronic inflammation resulting from chronic viral or bacteria infections. In particular, the methods described herein are useful for the treatment or amelioration of inflammatory disorders characterized by increased T cell activation and/or abnormal antigen presentation. The methods described herein can also be applied to the treatment or amelioration of one or more symptoms associated with inflammatory osteolysis, other disorders characterized by abnormal bone reabsorption, or disorders characterized by bone loss (e.g., osteoporosis).

The methods described herein are useful for the treatment, amelioration, or prevention of a transplant rejection including, but not limited to, a liver transplant rejection, a kidney transplant rejection, a bone transplant rejection, a skin transplant rejection, a heart transplant rejection, a blood transfusion rejection, and an eye transplant rejection.

The methods of the invention described herein can also be applied to skin conditions characterized by increased T cell or B cell activation and/or abnormal T cell or B cell activation such as, e.g., psoriasis, ultraviolet damage, atopic dermatitis, allergic and irritant contact dermatitis, lichen planus, alopecia areata, pyoderma gangrenosum, vitiligo, cicatrical pemphigoid, lupus erythematosus, scleroderma, and urticaria. Examples of the types of psoriasis which can be treated in accordance with the compositions and methods of the invention include, but are not limited to, plaque psoriasis, pustular psoriasis, erythrodermic psoriasis, guttate psoriasis and inverse psoriasis.

As used herein, the terms “immune-mediated disorder” and “immune-mediated disease” and analogous terms refer to disorders or diseases caused by the body's immune response. In a specific embodiment, an immune-mediated disorder is a disorder caused by an abnormal or uncontrolled T cell-mediated response. In another specific embodiment, the disorder is caused by an abnormal or uncontrolled B cell-mediated response. Examples of immune-mediated disorders include, but are not limited to, autoimmune disorders, inflammatory disorders, immune-mediated skin conditions, and transplant rejection. In certain embodiments, the immune-mediated disorder is not psoriasis or a hyperproliferative disorder. According to the present invention, the term “immune-mediated disorder” does not include cancer.

As used herein, the term “therapeutically effective amount” refers to that amount of the therapeutic agent sufficient to result in amelioration of one or more symptoms of a disorder, or prevent advancement of a disorder, or cause regression of the disorder. For example, with respect to the treatment of an inflammatory disorder or an autoimmune disorder characterized by inflammation, a therapeutically effective amount preferably refers to the amount of a therapeutic agent that reduces the inflammation of a joint, organ or tissue by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. With respect to the treatment of psoriasis, a therapeutically effective amount preferably refers to the amount of a therapeutic agent that reduces a human's Psoriasis Area and Severity Index (PASI) score by at least 20%, at least 35%, at least 30%, at least 40%, at least 45%, at least 50%, at least 55%, at least 60%, at least 65%, at least 70%, at least 75%, at least 80%, or at least 85%. Alternatively, with respect to the treatment of psoriasis, a therapeutically effective amount preferably refers to the amount of a therapeutic agent that improves a human's global assessment score by at least 25%, at least 35%, at least 30%, at least 40%, at least 45%, at least 50%, at least 55%, at least 60%, at least 65%, at least 70%, at least 75%, at least 80%, at least 85%, at least 90%, or at least 95%. With respect to the treatment of rheumatoid arthritis, a therapeutically effective amount preferably refers to the amount of a therapeutic agent that reduces a human's Disease Activity Score (DAS) score by at least 20%, at least 35%, at least 30%, at least 40%, at least 45%, at least 50%, at least 55%, at least 60%, at least 65%, at least 70%, at least 75%, at least 80%, or at least 85%. With respect to the treatment of systemic lupus erythematosus, a therapeutically effective amount preferably refers to the amount of a therapeutic agent that reduces a human's Systemic Lupus Activity Measure (SLAM) score by at least 20%, at least 35%, at least 30%, at least 40%, at least 45%, at least 50%, at least 55%, at least 60%, at least 65%, at least 70%, at least 75%, at least 80%, or at least 85%.

As used herein, the terms “transplant rejection,” “organ rejection,” and “graft-versus-host disease” are used interchangeably to refer to the rejection of a genetically non-identical tissue, organ, or graft by a recipient's immune system.

As used herein, the terms “treat,” “treatment,” and “treating” refer to the amelioration of one or more symptoms associated with an immune-mediated disorder that results from the administration of one or more therapeutic agents. In certain embodiments, such terms refer to a reduction in the swelling of one or more joints, or a reduction in the pain associated with an immune-mediated disorder resulting from the administration of one or more therapeutic agents to an animal with such a disorder. In other embodiments, such terms refer to a reduction in a human's PASI score, DAS score, or SLAM score. In other embodiments, such terms refer to an improvement in a human's global assessment score.

The term “active vitamin D compound,” as used herein, is intended to refer to a vitamin D compound that is or becomes biologically active (e.g., binds to and stimulates the vitamin D receptor) when administered to a subject or contacted with cells. Active vitamin D compounds include compounds that cause hypercalcemia and compounds that do not cause hypercalcemia upon administration. The biological activity of a vitamin D compound can be assessed by assays well known to one of skill in the art such as, e.g., immunoassays that measure the expression of a specific gene regulated by vitamin D. Vitamin D compounds exist in several forms with different levels of activity in the body. For example, a vitamin D compound may be partially activated by first undergoing hydroxylation in the liver at the carbon-25 position and then may be fully activated in the kidney by further hydroxylation at the carbon-1 position. The prototypical active vitamin D compound is 1α,25-hydroxyvitamin D₃, also known as calcitriol. A large number of other active vitamin D compounds are known and can be used in the practice of the invention. The active vitamin D compounds of the present invention include but are not limited to the analogs, homologs, mimics and derivatives of vitamin D compounds such as those described in the following patents, each of which is incorporated by reference: U.S. Pat. No. 4,391,802 (1α-hydroxyvitamin D derivatives); U.S. Pat. No. 4,717,721 (1α-hydroxy derivatives with a 17 side chain greater in length than the cholesterol or ergosterol side chains); U.S. Pat. No. 4,851,401 (cyclopentano-vitamin D analogs); U.S. Pat. Nos. 4,866,048 and 5,145,846 (vitamin D₃ analogues with alkynyl, alkenyl, and alkanyl side chains); U.S. Pat. No. 5,120,722 (trihydroxycalciferol); U.S. Pat. No. 5,547,947 (fluoro-cholecalciferol compounds); U.S. Pat. No. 5,446,035 (methyl substituted vitamin D); U.S. Pat. No. 5,411,949 (23-oxa-derivatives); U.S. Pat. No. 5,237,110 (19-nor-vitamin D compounds; U.S. Pat. No. 4,857,518 (hydroxylated 24-homo-vitamin D derivatives). Particular examples include ROCALTROL (Roche Laboratories); CALCIJEX injectable calcitriol; investigational drugs from Leo Pharmaceuticals including EB 1089 (24a,26a,27a-trihomo-22,24-diene-1α,25-(OH)₂-D₃, KH 1060 (20-epi-22-oxa-24a,26a,27a-trihomo-1α,25-(OH)₂-D₃), MC 1288 (1,25-(OH)₂-20-epi-D₃) and MC 903 (calcipotriol, 1α24s-(OH)₂-22-ene-26,27-dehydro-D₃); Roche Pharmaceutical drugs that include 1,25-(OH)₂-16-ene-D₃, 1,25-(OH)₂-16-ene-23-yne-D₃, and 25-(OH)₂-16-ene-23-yne-D₃; Chugai Pharmaceuticals 22-oxacalcitriol (22-oxa-1α,25-(OH)₂-D₃; 1α-(OH)-D₅ from the University of Illinois; and drugs from the Institute of Medical Chemistry-Schering AG that include ZK 161422 (20-methyl-1,25-(OH)₂-D₃) and ZK 157202 (20-methyl-23-ene-1,25-(OH)₂-D₃); 1α-(OH)-D₂; 1α-(OH)-D₃, 1α-(OH)-D₄, 25-(OH)-D₂; 25-(OH)-D₃; and 25-(OH)-D₄. Additional examples include 1α,25-(OH)₂-26,27-d₆-D₃; 1α,25-(OH)₂-22-ene-D₃; 1α,25-(OH)₂-D₃; 1α,25-(OH)₂-D₂; 1α,25-(OH)₂-D₄; 1α,24,25-(OH)₃-D₃; 1α,24,25-(OH)₃-D₂; 1α,25,25-(OH)₃-D₄; 1α-(OH)-25-FD₃; 1α-(OH)-25-FD₄; 1α-(OH)-25-FD₂; 1α,24-(OH)₂-D₄; 1α,24-(OH)₂-D₃; 1α,24-(OH)₂-D₂; 1α,24-(OH)₂-25-FD₄; 1α,24-(OH)₂-25-FD₃; 1α,24-(OH)₂-25-FD₂; 1α,25-(OH)₂-26,27-F₆-22-ene-D₃; 1α,25-(OH)₂-26,27-F₆-D_{3;} 1α,25S-(OH)₂-26-F₃-D₃; 1α,25-(OH)₂-24-F₂-D₃; 1α,25S,26-(OH)₂-22-ene-D_{3;}1α,25R,26-(OH)₂-22-ene-D₃; 1α,25-(OH)₂-D₂; 1α,25-(OH)₂-24-epi-D₃; 1α,25-(OH)₂-23-yne-D₃; 1α,25-(OH)₂-24R-F-D₃; 1α,25S,26-(OH)₂-D₃; 1α,24R-(OH)₂-25F-D₃; 1α,25-(OH)₂-26,27-F₆-23-yne-D₃; 1α,25R-(OH)₂-26-F₃-D₃; 1α,25,28-(OH)₃-D₂; 1α,25-(OH)₂-16-ene-23-yne-D₃; 1α,24R,25-(OH)₃-D_{3;} 1α,25-(OH)₂-26,27-F₆-23-ene-D₃; 1α,25R-(OH)₂-22-ene-26-F₃-D_{3;} 1α,25S-(OH)₂-22-ene-26-F₃-D₃; 1α,25R-(OH)₂-D₃-26,26,26-d₃; 1α,25S-(OH)₂-D₃-26,26,26-d₃; and 1α,25R-(OH)₂-22-ene-D₃-26,26,26-d₃. Additional examples can be found in U.S. Pat. No. 6,521,608. See also, e.g., U.S. Pat. Nos. 6,503,893, 6,482,812, 6,441,207, 6,410,523, 6,399,797, 6,392,071, 6,376,480, 6,372,926, 6,372,731, 6,359,152, 6,329,357, 6,326,503, 6,310,226, 6,288,249, 6,281,249, 6,277,837, 6,218,430, 6,207,656, 6,197,982, 6,127,559, 6,103,709, 6,080,878, 6,075,015, 6,072,062, 6,043,385, 6,017,908, 6,017,907, 6,013,814, 5,994,332, 5,976,784, 5,972,917, 5,945,410, 5,939,406, 5,936,105, 5,932,565, 5,929,056, 5,919,986, 5,905,074, 5,883,271, 5,880,113, 5,877,168, 5,872,140, 5,847,173, 5,843,927, 5,840,938, 5,830,885, 5,824,811, 5,811,562, 5,786,347, 5,767,111, 5,756,733, 5,716,945, 5,710,142, 5,700,791, 5,665,716, 5,663,157, 5,637,742, 5,612,325, 5,589,471, 5,585,368, 5,583,125, 5,565,589, 5,565,442, 5,554,599, 5,545,633, 5,532,228, 5,508,392, 5,508,274, 5,478,955, 5,457,217, 5,447,924, 5,446,034, 5,414,098, 5,403,940, 5,384,313, 5,374,629, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,804,502, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791.

The term “mimic” as used herein is intended to refer to non-secosteroidal vitamin D mimic compounds. In general, these non-secosteroidal vitamin D mimics are compounds that do not structurally fall within the class of compounds generally known as vitamin D compounds but which modulate the activity of vitamin D nuclear receptors. Examples of such vitamin D mimics include bis-aryl derivatives disclosed by U.S. Pat. No. 6,218,430 and WO publication 2005/037755. Additional examples of non-secosteroidal vitamin D mimic compounds suitable for the present invention can be found in U.S. Pat. Nos. 6,831,106; 6,706,725; 6,689,922; 6,548,715; 6,288,249; 6,184,422, 6,017,907, 6,858,595 and 6,358,939.

In one aspect the invention is drawn to methods employing non-secosteroidal vitamin D mimic compounds having Formula I:

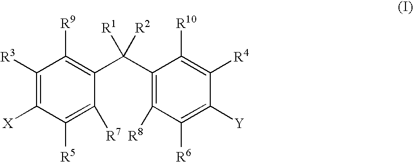

wherein:

R¹ and R² are each independently halo, haloalkyl, pseudohalo, optionally substituted alkyl, optionally substituted alkenyl, optionally substituted alkynyl, optionally substituted cycloalkyl, optionally substituted heterocyclyl, optionally substituted aryl or optionally substituted heteroaryl; or

R¹and R², together with the carbon atom to which they are attached, form an optionally substituted cycloalkyl consisting of:

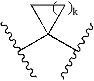

wherein k is an integer from 1 to 6; or

R¹ and R², together with the carbon atom to which they are attached, form an optionally substituted heterocyclyl selected from a group consisting of:

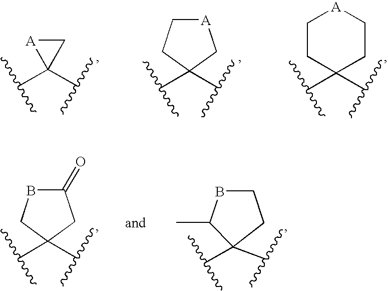

wherein A is —O—, —NR^{x}—, —S—, —S(O)— or —S(O)₂— wherein R^{x} is hydrogen, alkyl, haloalkyl, cycloalkyl, heterocyclyl, aryl, heteroaryl, —R¹⁴—C(J)R¹⁵, —R¹⁴—C(J)OR¹⁵, —R¹⁴—C(J)R¹⁶OR¹⁵, —R¹⁴—C(J)SR¹⁶, —R¹⁴—C(J)N(R¹⁸)R¹⁹, —R¹⁴—C(J)N(R¹⁷)N(R¹⁸)R¹⁹, —R¹⁴—C(J)N(R¹⁷)S(O)ₚR²⁰, —R¹⁴—S(O)ₚN(R¹⁸)R¹⁹, or —R¹⁴—S(O)ₚR²⁰; and wherein B is —O—, —S— or —NR^{y} where R^{y} is hydrogen, alkyl, haloalkyl, aryl or heteroaryl; and wherein each p is independently 0 to 2;

R³ and R⁴ are each independently hydrogen, alkyl, alkenyl, alkynyl, cycloalkyl, heterocyclyl, aryl, heteroaryl, halo, pseudohalo, nitro, cyano, azido, —R¹⁴—OR¹⁵, —R¹⁴—N(R¹⁸)R¹⁹, —R¹⁴—SR¹⁵, —R¹⁴—OC(J)R¹⁵, —R¹⁴—NR¹⁷C(J)R¹⁵, —R¹⁴—OC(J)N(R¹⁸)R¹⁹, —R¹⁴—NR¹⁷C(J)N(R¹⁸)R¹⁹, —R¹⁴—NR¹⁷C(J)OR¹⁵, —R¹⁴—C(J)R¹⁵, —R¹⁴—C(J)OR¹⁵, —R¹⁴—C(J)SR¹⁵, —R¹⁴—C(J)N(R¹⁸)R¹⁹, or —R¹⁴—C(J)N(R¹⁷)N(R¹⁸)R¹⁹;

R⁵, R⁶, R⁷, R⁸, R⁹, R¹⁰ are each independently hydrogen, halo, hydroxy, amino, pseudohalo, cyano, nitro, alkyl, haloalkyl, alkoxy or haloalkoxy;

X is R²⁵;

Y is independently R³⁰, —OR³¹, —SR³² or —N(R³³)(R³⁴);

R²⁵ and R³⁰ are each independently selected from (i) or (ii) as follows:

(i) optionally substituted alkyl that may be substituted with one to ten substituents each independently selected from a group consisting of halo, pseudohalo, nitro, cyano, thioxo, azido, amidino, guanidino, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OR¹⁵, —OR¹⁶OR¹⁵, —N(R¹⁸)R¹⁹, —N(R¹⁷)N(R¹⁸)R¹⁹, —SR¹⁵, —SR¹⁶SR¹⁵, —N(R¹⁷)N(R¹⁷)S(O)ₚR²⁰, —OC(J)R¹⁵, —NR¹⁷C(J)R¹⁵, —OC(J)N(R¹⁸)R¹⁹, —NR¹⁷C(J)N(R¹⁸)R¹⁹, —NR¹⁷C(J)OR¹⁵, —OC(J)OR¹⁵, —P(R²¹)₂, —P(O)(R²¹)₂, —OP(O)(R²¹)₂, —C(J)R¹⁵, —C(J)OR¹⁵, —C(J)SR¹⁶, —C(J)(R¹⁸)R¹⁹, —C(J)N(R¹⁷)N(R¹⁸)R¹⁹, —C(J)N(R¹⁷)N(R¹⁷)S(O)ₚR²⁰, —C(R¹⁷)═NOR¹⁵, —C(R¹⁷)═NR¹⁷, —C(R¹⁷)═NN(R¹⁸)R¹⁹ and —C(═NR¹⁷)N(R¹⁸)R¹⁹; or

(ii) optionally substituted alkenyl or optionally substituted alkynyl, either of which may be substituted with one to ten substituents each independently selected from a group consisting of oxo, thioxo, halo, pseudohalo, nitro, cyano, azido, amidino, guanidino, —OR¹⁵, —OR¹⁶OR¹⁵, —N(R¹⁸)R¹⁹, —N(R¹⁷)N(R¹⁸)R¹⁹, —SR¹⁵, —SR¹⁶SR¹⁵, —S(O)ₚR²⁰, —N(R¹⁷)S(O)ₚR²⁰, —N(R¹⁷)N(R¹⁷)S(O)ₚR²⁰, —OC(J)R¹⁵, —NR¹⁷C(J)R¹⁵, —OC(J)N(R¹⁸)R¹⁹, —NR¹⁷C(J)N(R¹⁸)R¹⁹, —NR¹⁷C(J)OR¹⁵, —OC(J)OR¹⁵, —P(R²¹)₂, —P(O)(R²¹)₂, —OP(O)(R²¹)₂, —C(J)R¹⁵, —C(J)OR¹⁵, —C(J)SR¹⁶, —C(J)N(R¹⁸)R¹⁹, —C(J)N(R¹⁷)N(R¹⁸)R¹⁹, —C(J)N(R¹⁷)S(O)ₚR²⁰, —C(J)N(R¹⁷)N(R¹⁷)S(O)ₚR²⁰, —C(R¹⁷)═NOR¹⁵, —C(R¹⁷)═NR¹⁷, —C(R¹⁷)═NN(R¹⁸)R¹⁹, —C(═NR¹⁷)N(R¹⁸)R¹⁹, alkyl, haloalkyl, cycloalkyl, heterocyclyl, aryl and heteroaryl;

R^{31,} R³², R³³, and R³⁴ are each independently optionally substituted alkyl, optionally substituted alkenyl, optionally substituted alkynyl or optionally substituted cycloalkyl; all of which may be optionally substituted with one to ten substituents each independently selected from a group consisting of oxo, halo, pseudohalo, nitro cyano, azido, amidino, guanidino —OR¹⁵, —OR¹⁶OR¹⁵, —N(R¹⁸)R¹⁹, —N(R¹⁷)N(R¹⁸)R¹⁹, —SR¹⁵, —SR¹⁶SR¹⁵, —S(O)ₚR²⁰, —N(R¹⁷)S(O)ₚR²⁰, —N(R¹⁷)N(R¹⁷)S(O)ₚR²⁰, —OC(J)R¹⁵, —NR¹⁷C(J)R¹⁵, —OC(J)N(R¹⁸)R¹⁹, —NR¹⁷C(J)N(R¹⁸)R¹⁹, —NR¹⁷C(J)OR¹⁵, —OC(J)OR¹⁵, —P(R²¹)₂, —P(O)(R²¹)₂, —OP(O)(R²¹)₂, —C(J)R¹⁵, —C(J)OR¹⁵, —C(J)SR¹⁶, —C(J)N(R¹⁸)R¹⁹, —C(J)N(R¹⁷)N(R¹⁸)R¹⁹, —C(J)N(R¹⁷)S(O)ₚR²⁰, —C(J)N(R¹⁷)N(R¹⁷)S(O)ₚR²⁰, —C(R¹⁷)═NOR¹⁵, —C(R¹⁷)═NR¹⁷, —C(R¹⁷)═NN(R¹⁸)R¹⁹, —C(═NR¹⁷)N(R¹⁸)R¹⁹, alkyl, cycloalkyl, heterocyclyl, aryl and heteroaryl, and R³⁴ can additionally be hydrogen;

where each R¹⁴is independently a direct bond or alkylene;

where each R¹⁵ and R¹⁷ is independently hydrogen, optionally substituted alkyl, optionally substituted alkenyl, optionally substituted alkynyl, optionally substituted cycloalkyl, optionally substituted heterocyclyl, optionally substituted aryl or optionally substituted heteroaryl, all of which, when substituted, are substituted with one to five substituents each independently selected from halo, cyano, hydroxy and amino;

where each R¹⁶ and R²⁰ is independently optionally substituted alkyl, optionally substituted alkenyl, optionally substituted alkynyl, optionally substituted cycloalkyl, optionally substituted heterocyclyl, optionally substituted aryl or optionally substituted heteroaryl, all of which, when substituted, are substituted with one to five substituents each independently selected from halo, hydroxy, alkoxy and amino; and

where each R¹⁸ and R¹⁹ is independently hydrogen, optionally substituted alkyl, optionally substituted alkenyl, optionally substituted alkynyl, optionally substituted cycloalkyl, optionally substituted heterocyclyl, optionally substituted aryl or optionally substituted heteroaryl, all of which, when substituted, are substituted with one to five substituents each independently selected from halo, hydroxy, alkoxy and amino;

or where R¹⁸ and R¹⁹, together with the nitrogen atom to which they are attached, form a heterocyclyl or heteroaryl;

each R²¹ is independently alkyl, —OR²² or —N(R²³)R²⁴;

R²² is hydrogen, alkyl, haloalkyl, alkenyl, alkynyl, cycloalkyl, heterocyclyl, aryl, heteroaryl or aralkyl;

R²³ and R²⁴ are each independently hydrogen, alkyl, haloalkyl, alkenyl, alkynyl or cycloalkyl;

or R²³ and R²⁴, together with the nitrogen atom to which they are attached, form a heterocyclyl or heteroaryl;

each J is independently O or S;

as a single isomer, a mixture of isomers, or as a racemic mixture of isomers; as a solvate or polymorph; or as a prodrug or metabolite; or as a pharmaceutically acceptable salt thereof.

In one embodiment, R¹ and R² may form a substituted cyclohexyl, said cyclohexyl, when substituted at the 4-position relative to the gem-diaryl substituents, may be substituted with a substituent selected from the group consisting of halo, cyano, optionally substituted alkyl, optionally substituted alkenyl, optionally substituted alkynyl, optionally substituted aryl and optionally substituted heteroaryl.

In another embodiment, R²⁵ and R³⁰ are not —CH₂COOH; —CH₂-5-tetrazolyl; —CH₂COOMe; —CH₂COOEt; —CH₂NH(CH₂COOH); —CH₂N(C(O)Me)(CH₂COOH); —CH₂—N-pyrrolidin-2-one; —CH₂-(1-methylpyrrolidin-2-one-3-yl); —CH₂C(O)NH₂; —CH₂C(O)NMe₂; —CH₂C(O)NHMe; —CH₂C(O)—N-pyrrolidone; —CH(OH)COOH; —CH(OH)C(O)NH₂; —CH(OH)C(O)NHMe; —CH(OH)C(O)NMe₂; —CH(OH)C(O)NEt₂; —CH₂CH₂COOH; —CH₂CH₂COOMe; —CH₂CH₂COOEt; —CH₂CH₂C(O)NH₂; —CH₂CH₂C(O)NHMe; —CH₂CH₂C(O)NMe₂; or —CH₂CH₂-5-tetrazolyl.

In another aspect the invention is drawn to methods employing the following non-secosteroidal vitamin D mimic compounds:

3-(2-methyl-4-{2,2,2-trifluoro-1-[4-(2-hydroxy-3,3-dimethyl-butoxy)-3-methyl-phenyl]-1-phenyl-ethyl}-phenoxy)-propane-1,2-diol;

3-(4-{4-[4-(2-hydroxy-3,3-dimethyl-butoxy)-3-methyl-phenyl]-piperidin-4-yl}-2-methyl-phenoxy)-propane-1,2-diol;

3-(4-{4-[4-(2-hydroxy-3,3-dimethyl-butoxy)-3-methyl-phenyl]-piperidin-4-yl}-2-methyl-phenoxy)-propane-1,2(S)-diol;

1-{4-[4-(2(S),3-dihydroxy-propoxy)-3-methyl-phenyl]-4-[4-(2-hydroxy-3,3-dimethyl-butoxy)-3-methyl-phenyl]-piperidin-1-yl}-ethanone;

1-(4-{1-acetyl-4-[4-(3,3-dimethyl-2-oxo-butoxy)-3-methyl-phenyl]-piperidin-4-yl}-2-methyl-phenoxy)-3,3-dimethyl-butan-2-one;

3-(4-{1-ethyl-1-[4-(3-hydroxy-3-methylbutyl)-3-methylphenyl]-propyl}-2-methylphenoxy)-propane-1,2(S)-diol;

3-(4-{1-ethyl-1-[4-(3-ethyl-3-hydroxypentyl)-3-methylphenyl]-propyl}-2-methyl-phenoxy)-propane-1,2(S)-diol;

3-(4-{1-ethyl-1-[4-(3-hydroxy-5-methylhexyl)-3-methylphenyl]-propyl}-2-methyl-phenoxy)-propane-1,2(S)-diol;

3-(4-{1-ethyl-1-[4-(3-hydroxy-4-methylpentyl)-3-methylphenyl]-propyl}-2-methyl-phenoxy)-propane-1,2(S)-diol;

3-(2-ethyl-4-{1-ethyl-1-[4-(3-hydroxy-4,4-dimethylpentyl)-3-methylphenyl]-propyl}-phenoxy)-propane-1,2(S)-diol;

3-(4-{1-ethyl-1-[4-(3-hydroxy-4,4-dimethylpentyl)-3-methylphenyl]-propyl}-2-methyl-phenoxy)-propane-1,2(S)-diol;

3-[4-(1-ethyl-1-{4-[3(S)-hydroxy-4,4-dimethylpentyl]-3-methylphenyl}-propyl)-2-methyl-phenoxy]-propane-1,2(S)-diol;

3-[4-(1-ethyl-1-{4-[3(R)-hydroxy-4,4-dimethylpentyl]-3-methylphenyl}-propyl)-2-methyl-phenoxy]-propane-1,2(S)-diol and

3-(4-{1-ethyl-1-[4-(3-hydroxy-4,4-dimethylpentyl)-phenyl]-propyl}-2-methylphenoxy)-propane-1,2(S)-diol.

In another aspect the invention is drawn to methods employing non-secosteroidal vitamin D mimic compounds having Formula II:

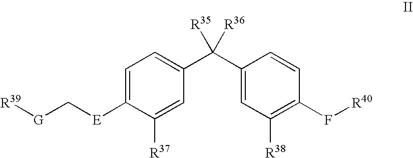

wherein:

E and F are each independently selected from the group consisting of O, S, and NR⁴¹;

G is selected from the group consisting of C═O, CH(OR⁴²), and CH(NR⁴³R⁴⁴);

R³⁵ and R³⁶ are independently selected from the group consisting of alkyl groups, optionally fluorinated; or together R³⁵ and R³⁶ form a cycloalkylidene having 3 to 8 carbon atoms, optionally fluorinated;

R³⁷ and R³⁸ are independently selected from the group consisting of halogen; lower n-alkyl, optionally fluorinated; and lower alkoxy, optionally fluorinated;

R³⁹ is selected from the group consisting of H; optionally substituted alkyl groups; optionally substituted alkenyl groups; optionally substituted alkynyl groups; optionally substituted aryl groups; OR⁴⁵; NR⁴⁶R⁴⁷; or together with R⁴², R⁴³, or R⁴⁴ forms a 3- to 12-membered cyclic group wherein said cyclic group is selected from the group consisting of amidines, amines, ethers, lactams, lactones, ketals, hemiketals, aminals, hemiaminals, carbonates, carbamates, ureas, and combinations thereof;

R⁴⁰ is selected from the group consisting of H and alkyl groups, optionally substituted;

R⁴¹ is selected from the group consisting of H and alkyl groups, optionally substituted;

R⁴² is selected from the group consisting of H, optionally substituted alkyl groups, optionally substituted alkenyl groups, optionally substituted alkynyl groups, optionally substituted aryl group, and optionally substituted acyl groups;

R⁴³ and R⁴⁴ are independently selected from the group consisting of H, optionally substituted alkyl groups, optionally substituted alkenyl groups, optionally substituted alkynyl groups, optionally substituted aryl groups, and optionally substituted acyl groups;

R⁴⁵ is selected from the group consisting of H, optionally substituted alkyl groups, optionally substituted alkenyl groups, optionally substituted alkynyl groups, optionally substituted aryl groups, and optionally substituted acyl groups; and

R⁴⁶ and R⁴⁷ are independently selected from the group consisting of H, optionally substituted alkyl groups, optionally substituted alkenyl groups, optionally substituted alkynyl groups, optionally substituted aryl groups, and optionally substituted acyl groups and pharmaceutically acceptable salts thereof.

In a first embodiment, when K and L are both O, M is C═O, and R⁴⁵ is selected from the group consisting of OH and C₁-C₄ alkoxy, then R⁴⁶ is not carboxymethyl and alkyl esters thereof. In a second embodiment, when K and L are both O, and M is selected from the group consisting of CH(OR⁴⁸) and CH(NR⁴⁹R⁵⁰), then R⁴⁵ is not H or primary alkyl. In a third embodiment, when K and L are both O, and M is CH(OR⁴⁸), then R⁴⁶ and R⁴⁸ do not both comprise aziridines. In a fourth embodiment, when K and L are both O, and M is CH(OR⁴⁸), then R⁴⁵, R⁴⁶, and R⁴⁸ do not simultaneously comprise alkenyl ethers. In a fifth embodiment, when K and L are both O, and M is CH(OR⁴⁸), then R⁴⁵ and R⁴⁶ do not both comprise glycidyl ethers.

In a preferred embodiment of the invention, the active vitamin D compound has a reduced hypercalcemic effect as compared to vitamin D so that sufficient doses of the compound can be administered without inducing hypercalcemia in the animal. The reduced hypercalcemic effect may be due to the active vitamin D compound itself, the regimen by which the compound is administered, or both. A reduced hypercalcemic effect is defined as an effect which is less than the hypercalcemic effect induced by administration of an equal dose of 1α,25-hydroxyvitamin D₃ (calcitriol). As an example, EB 1089 has a hypercalcemic effect which is 50% of the hypercalcemic effect of calcitriol. Additional active vitamin D compounds having a reduced hypercalcemic effect include Ro23-7553 and Ro24-5531 available from Hoffman LaRoche. Other examples of active vitamin D compounds having a reduced hypercalcemic effect can be found in U.S. Pat. No. 4,717,721. Determining the hypercalcemic effect of an active vitamin D compound is routine in the art and can be carried out as disclosed in Hansen et al., Curr. Pharm. Des. 6:803-828 (2000).

Administration of Active Vitamin D Compounds

The active vitamin D compound or a mimic thereof is preferably administered at a dose of about 0.1 μg to about 10 mg, e.g., about 0.5 μg to about 1 mg, or from about 15 μg to about 500 μg. In a specific embodiment, an effective amount of an active vitamin D compound or a mimic thereof is 0.5, 1, 2, 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, 450, 500, 550, 600, 650, 700, 750, 800, 850, 900, 950, 1000, 2000, 3000, 4000, 5000, 6000, 7000, 8000, 9000, or 10000 μg or more. In certain embodiments, an effective dose of an active vitamin D compound or a mimic thereof is between about 3 μg to about 10 mg, e.g., between about 15 μg to about 1 mg, between about 30 μg to about 300 μg, between about 35 μg to about 200 μg, between about 40 μg to about 100 μg, or about 45 μg. In certain embodiments, the methods of the invention comprise administering an active vitamin D compound or a mimic thereof in a dose of about 0.12 μg/kg bodyweight to about 200 μg/kg bodyweight. The compound may be administered by any route, including oral, intramuscular, intravenous, parenteral, rectal, nasal, topical, or transdermal.

If the active vitamin D compound or a mimic thereof is to be administered daily, the dose may be kept low, for example about 0.5 μg to about 5 μg, in order to avoid or diminish the induction of hypercalcemia. If the active vitamin D compound or a mimic thereof has a reduced hypercalcemic effect a higher daily dose may be administered without resulting in hypercalcemia, for example about 10 μg to about 20 μg or higher (up to about 50 μg to about 100 μg).

In a preferred embodiment of the invention, the active vitamin D compound or a mimic thereof is administered in a pulsed-dose fashion so that high doses of the active vitamin D compound or a mimic thereof can be administered without inducing hypercalcemia. Pulsed dosing refers to intermittently administering an active vitamin D compound or a mimic thereof on either a continuous intermittent dosing schedule or a non-continuous intermittent dosing schedule. High doses of active vitamin D compounds or a mimic thereof include doses greater than about 3 μg as discussed in the sections above. Therefore, in certain embodiments of the invention, the methods for the treatment or amelioration of immune-mediated disorders encompass intermittently administering high doses of active vitamin D compounds or a mimic thereof. The frequency of the pulsed-dose administration can be limited by a number of factors including, but not limited to, the pharmacokinetic parameters of the compound or formulation and the pharmacodynamic effects of the active vitamin D compound or a mimic thereof on the animal. For example, animals with immune-mediated disorders having impaired renal function may require less frequent administration of the active vitamin D compound or a mimic thereof because of the decreased ability of those animals to excrete calcium.

The following is exemplary only and merely serves to illustrate that the term “pulsed-dose” can encompass any discontinuous administration regimen designed by a person of skill in the art.

In one example, the active vitamin D compound or a mimic thereof can be administered not more than once every three days, every four days, every five days, every six days, every seven days, every eight days, every nine days, every ten days, every two weeks, every three weeks, or every four weeks. The administration can continue for one, two, three, or four weeks or one, two, or three months, or longer. Optionally, after a period of rest, the active vitamin D compound or a mimic thereof can be administered under the same or a different schedule. The period of rest can be one, two, three, or four weeks, or longer, according to the pharmacodynamic effects of the active vitamin D compound or a mimic thereof on the animal.

In another example, the active vitamin D compound or a mimic thereof can be administered once per week for three months.

In a preferred embodiment, the vitamin D compound or a mimic thereof can be administered once per week for three weeks of a four week cycle. After a one week period of rest, the active vitamin D compound can be administered under the same or different schedule.

Further examples of dosing schedules that can be used in the methods of the present invention are provided in published U.S. Pat. No. 6,521,608, which is incorporated by reference in its entirety.

The above-described administration schedules are provided for illustrative purposes only and should not be considered limiting. A person of skill in the art will readily understand that all active vitamin D compounds or mimics thereof are within the scope of the invention and that the exact dosing and schedule of administration of the active vitamin D compounds or mimics thereof can vary due to many factors.

The amount of a therapeutically effective dose of a pharmaceutical agent in the acute or chronic management of a disease or disorder may differ depending on factors including but not limited to the disease or disorder treated, the specific pharmaceutical agents and the route of administration. According to the methods of the invention, an effective dose of an active vitamin D compound or a mimic thereof is any dose of the compound effective to treat or ameliorate immune-mediated disorders. A high dose of an active vitamin D compound can be a dose from about 3 μg to about 10 mg or any dose within this range as discussed above. The dose, dose frequency, duration, or any combination thereof, may also vary according to age, body weight, response, and the past medical history of the animal as well as the route of administration, pharmacokinetics, and pharmacodynamic effects of the pharmaceutical agents. These factors are routinely considered by one of skill in the art.

The rate of absorption and clearance of vitamin D compounds or mimics thereof are affected by a variety of factors that are well known to persons of skill in the art. As discussed above, the pharmacokinetic properties of active vitamin D compounds limit the peak concentration of vitamin D compounds that can be obtained in the blood without inducing the onset of hypercalcemia. The rate and extent of absorption, distribution, binding or localization in tissues, biotransformation, and excretion of the active vitamin D compound can all affect the frequency at which the pharmaceutical agents can be administered. In certain embodiments, active vitamin D compounds are administered in a pulsed-dose fashion in high doses as a method of treating or ameliorating immune-mediated disorders according to the dosing schedule described above.

In one embodiment of the invention, an active vitamin D compound or a mimic thereof is administered at a dose sufficient to achieve peak plasma concentrations of the active vitamin D compound of about 0.1 nM to about 1000 nM, e.g., about 0.1 nM to about 20 nM. In certain embodiments, the methods of the invention comprise administering the active vitamin D compound in a dose that achieves peak plasma concentrations of 0.1 nM, 0.2 nM,40.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 12.5 nM, 15 nM, 17.5 nM 20 nM, 22.5 nM, 25 nM, 30 nM, 35 nM, 40 nM, 45 nM, 50 nM, 60 nM, 70 nM, 80 nM, 90 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM or 1000 nM or any range of concentrations therein. In other embodiments, the active vitamin D compound is administered in a dose that achieves peak plasma concentrations of the active vitamin D compound exceeding about 0.5 nM, e.g., about 0.5 nM to about 1000 nM, about 0.5 nM to about 100 nM, about 0.5 nM to about 20 nM about 1 nM to about 10 nM, about 1 nM to about 7 nM, or about 3 nM to about 5 nM.

In another preferred embodiment, the active vitamin D compound or a mimic thereof is administered at a dose of at least about 0.12 μg/kg bodyweight, more preferably at a dose of at least about 0.5 μg/kg bodyweight.

One of skill in the art will recognize that these standard doses are for an average sized adult of approximately 70 kg and can be adjusted for the factors routinely considered as stated above.

In certain embodiments, the methods of the invention further comprise administering a dose of an active vitamin D compound or a mimic thereof that achieves peak plasma concentrations rapidly, e.g., within four hours. In further embodiments, the methods of the invention comprise administering a dose of an active vitamin D compound that is eliminated quickly, e.g., with an elimination half-life of less than 12 hours.

While obtaining high concentrations of the active vitamin D compound or a mimic thereof is beneficial, it must be balanced with clinical safety, e.g., hypercalcemia. Thus, in one aspect of the invention, the methods of the invention encompass intermittently administering high doses of active vitamin D compounds or mimics thereof to an animal with an immune-mediated disorder and monitoring the animal for symptoms associated with hypercalcemia. Such symptoms include calcification of soft tissues (e.g., cardiac tissue), increased bone density, and hypercalcemic nephropathy. In still another embodiment, the methods of the invention encompass intermittently administering high doses of an active vitamin D compound or a mimic thereof to an animal with an immune-mediated disorder and monitoring the calcium plasma concentration of the animal to ensure that the calcium plasma concentration is less than about 11.5 mg/dL.

In certain embodiments, high blood levels of vitamin D compounds or mimics thereof can be safely obtained in conjunction with reducing the transport of calcium into the blood. In one embodiment, higher active vitamin D compound concentrations are safely obtainable without the onset of hypercalcemia when administered in conjunction with a reduced calcium diet, e.g., a calcium intake of less than 600 mg/day, preferably about 400 to about 500 mg/day. In another embodiment, the active vitamin D compound or a mimic thereof may be administered at night before bedtime to minimize calcium absorption. See U.S. Pat. No. 5,891,865. In one example, the calcium can be trapped by an adsorbent, absorbent, ligand, chelate, or other binding moiety that cannot be transported into the blood through the small intestine. In another example, the rate of osteoclast activation can be inhibited by administering, for example, a bisphosphonate such as, e.g., zoledronate, pamidronate, or alendronate in conjunction with the active vitamin D compound.

In certain embodiments, high blood levels of active vitamin D compounds are safely obtained in conjunction with maximizing the rate of clearance of calcium. In one example, calcium excretion can be increased by ensuring adequate hydration and salt intake. In another example, diuretic therapy can be used to increase calcium excretion.

The doses of the vitamin D analogs and vitamin D mimics may be adjusted proportionate to the ratio of the efficacy index to the calcemic index according to the formula:
Dose=CalcitriolDose×(EI÷CI)
where Dose is the analog or mimic dose, calcitriolDose is calcitriol dose, EI is the analog or mimic efficacy index and CI is the analog or mimic calcemic index, wherein the term “efficacy index” is the ratio of the concentration of the vitamin D analog or mimic to the concentration of calcitriol at equivalent potency. Thus, the efficacy index is a fraction less than one when the vitamin D analog or mimic is less potent than calcitriol. EI is a number greater than one when calcitriol is less potent than the vitamin D analog or mimic. The “calcemic index” of a drug is a measure of the relative ability of the drug to generate a calcemic response as reported in Bouillon et al., Endocrine Rev. 16:200 (1995). A calcemic index of 1 corresponds to the relative calcemic activity of calcitriol. A calcemic index of about 0.01 corresponds to the calcemic activity of a drug with approximately 100 times less calcemic activity than calcitriol. A calcemic index of 0.5 would correspond to a drug having approximately half the calcemic activity of calcitriol. The calcemic index of a drug can vary depending on the assay conducted, e.g., whether one is measuring stimulation of intestinal calcium absorption (a process by which dietary calcium enters into the physiological processes to contribute to the skeletal growth of the organism and to the maintenance of calcium homeostasis) or bone calcium mobilizing activity (a process by which the bone matrix acts as an exchangeable reservoir for calcium). See U.S. Pat. No. 6,521,608 for further detail.

The active vitamin D compound or a mimic thereof may be administered as part of a pharmaceutical composition comprising a pharmaceutically acceptable carrier, wherein the active vitamin D compound or a mimic thereof is present in an amount which is effective to achieve its intended purpose. The pharmaceutical composition may further comprise one or more excipients, diluents or any other components known to persons of skill in the art and germane to the methods of formulation of the present invention.

The pharmaceutical composition can be prepared in single unit dosage forms. The dosage forms are suitable for oral, mucosal (nasal, sublingual, vaginal, buccal, rectal), parenteral (intravenous, intramuscular, intraarterial), or topical administration. Preferred dosage forms of the present invention include oral dosage forms and intravenous dosage forms.

Intravenous forms include, but are not limited to, bolus and drip injections. In preferred embodiments, the intravenous dosage forms are sterile or capable of being sterilized prior to administration to a subject since they typically bypass the subject's natural defenses against contaminants. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

In a preferred embodiment of the invention, the pharmaceutical compositions comprising active vitamin D compounds or mimics thereof are emulsion pre-concentrate formulations. The compositions of the invention meet or substantially reduce the difficulties associated with active vitamin D compound therapy hitherto encountered in the art including, in particular, undesirable pharmacokinetic parameters of the compound upon administration to a patient.

According to one aspect of the present invention, a pharmaceutical composition is provided comprising (a) a lipophilic phase component, (b) one or more surfactants, (c) an active vitamin D compound; wherein said composition is an emulsion pre-concentrate, which upon dilution with water, in a water to composition ratio of about 1:1 or more of said water, forms an emulsion having an absorbance of greater than 0.3 at 400 nm. The pharmaceutical composition of the invention may further comprise a hydrophilic phase component.

In another aspect of the invention, a pharmaceutical emulsion composition is provided comprising water (or other aqueous solution) and an emulsion pre-concentrate.

The term “emulsion pre-concentrate,” as used herein, is intended to mean a system capable of providing an emulsion upon contacting with, e.g., water. The term “emulsion,” as used herein, is intended to mean a colloidal dispersion comprising water and organic components including hydrophobic (lipophilic) organic components. The term “emulsion” is intended to encompass both conventional emulsions, as understood by those skilled in the art, as well as “sub-micron droplet emulsions,” as defined immediately below.

The term “sub-micron droplet emulsion,” as used herein is intended to mean a dispersion comprising water and organic components including hydrophobic (lipophilic) organic components, wherein the droplets or particles formed from the organic components have an average maximum dimension of less than about 1000 nm.

Sub-micron droplet emulsions are identifiable as possessing one or more of the following characteristics. They are formed spontaneously or substantially spontaneously when their components are brought into contact, that is without substantial energy supply, e.g., in the absence of heating or the use of high shear equipment or other substantial agitation. They exhibit thermodynamic stability and they are monophasic.

The particles of a sub-micron droplet emulsion may be spherical, though other structures are feasible, e.g. liquid crystals with lamellar, hexagonal or isotropic symmetries. Generally, sub-micron droplet emulsions comprise droplets or particles having a maximum dimension (e.g., average diameter) of between about 50 nm to about 1000 nm, and preferably between about 200 nm to about 300 nm.

The term “pharmaceutical composition” as used herein is to be understood as defining compositions of which the individual components or ingredients are themselves pharmaceutically acceptable, e.g., where oral administration is foreseen, acceptable for oral use and, where topical administration is foreseen, topically acceptable.

The pharmaceutical compositions of the present invention will generally form an emulsion upon dilution with water. The emulsion will form according to the present invention upon the dilution of an emulsion pre-concentrate with water in a water to composition ratio of about 1:1 or more of said water. According to the present invention, the ratio of water to composition can be, e.g., between 1:1 and 5000:1. For example, the ratio of water to composition can be about 1:1, 2:1, 3:1, 4:1, 5:1, 10:1, 200:1, 300:1, 500:1, 1000:1, or 5000:1. The skilled artisan will be able to readily ascertain the particular ratio of water to composition that is appropriate for any given situation or circumstance.

According to the present invention, upon dilution of said emulsion pre-concentrate with water, an emulsion will form having an absorbance of greater than 0.3 at 400 nm. The absorbance at 400 nm of the emulsions formed upon 1:100 dilution of the emulsion pre-concentrates of the present invention can be, e.g., between 0.3 and 4.0. For example, the absorbance at 400 nm can be, e.g., about 0.4, 0.5, 0.6, 1.0, 1.2, 1.6, 2.0, 2.2, 2.4, 2.5, 3.0, or 4.0. Methods for determining the absorbance of a liquid solution are well known by those in the art. The skilled artisan will be able to ascertain and adjust the relative proportions of the ingredients of the emulsions pre-concentrates of the invention in order to obtain, upon dilution with water, an emulsion having any particular absorbance encompassed within the scope of the invention.

The pharmaceutical compositions of the present invention can be, e.g., in a semi-solid formulation or in a liquid formulation. Semi-solid formulations of the present invention can be any semi-solid formulation known by those of ordinary skill in the art, including, e.g., gels, pastes, creams and ointments.

The pharmaceutical compositions of the present invention comprise a lipophilic phase component. Suitable components for use as lipophilic phase components include any pharmaceutically acceptable solvent which is non-miscible with water. Such solvents will appropriately be devoid or substantially devoid of surfactant function.

The lipophilic phase component may comprise mono-, di- or triglycerides. Mono-, di- and triglycerides that may be used within the scope of the invention include those that are derived from C₆, C₈, C₁₀, C₁₂, C₁₄, C₁₆, C₁₈, C₂₀ and C₂₂ fatty acids. Exemplary diglycerides include, in particular, diolein, dipalmitolein, and mixed caprylin-caprin diglycerides. Preferred triglycerides include vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides, and mixtures thereof.

Among the above-listed triglycerides, preferred triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate.

A preferred triglyceride is the medium chain triglyceride available under the trade name LABRAFAC CC. Other preferred triglycerides include neutral oils, e.g., neutral plant oils, in particular fractionated coconut oils such as known and commercially available under the trade name MIGLYOL, including the products: MIGLYOL 810; MIGLYOL 812; MIGLYOL 818; and CAPTEX 355.

Also suitable are caprylic-capric acid triglycerides such as known and commercially available under the trade name MYRITOL, including the product MYRITOL 813. Further suitable products of this class are CAPMUL MCT, CAPTEX 200, CAPTEX 300, CAPTEX 800, NEOBEE M5 and MAZOL 1400.

Especially preferred as lipophilic phase component is the product MIGLYOL 812. (See U.S. Pat. No. 5,342,625).

Pharmaceutical compositions of the present invention may further comprise a hydrophilic phase component. The hydrophilic phase component may comprise, e.g., a pharmaceutically acceptable C₁₋₅ alkyl or tetrahydrofurfuryl di- or partial-ether of a low molecular weight mono- or poly-oxy-alkanediol. Suitable hydrophilic phase components include, e.g., di- or partial-, especially partial-, -ethers of mono- or poly-, especially mono- or di-, -oxy-alkanediols comprising from 2 to 12, especially 4 carbon atoms. Preferably the mono- or poly-oxy-alkanediol moiety is straight-chained. Exemplary hydrophilic phase components for use in relation to the present invention are those known and commercially available under the trade names TRANSCUTOL and COLYCOFUROL. (See U.S. Pat. No. 5,342,625).

In an especially preferred embodiment, the hydrophilic phase component comprises 1,2-propyleneglycol.

The hydrophilic phase component of the present invention may of course additionally include one or more additional ingredients. Preferably, however, any additional ingredients will comprise materials in which the active vitamin D compound is sufficiently soluble, such that the efficacy of the hydrophilic phase as an active vitamin D compound carrier medium is not materially impaired. Examples of possible additional hydrophilic phase components include lower (e.g., C₁₋₅) alkanols, in particular ethanol.

Pharmaceutical compositions of the present invention also comprise one or more surfactants. Surfactants that can be used in conjunction with the present invention include hydrophilic or lipophilic surfactants, or mixtures thereof. Especially preferred are non-ionic hydrophilic and non-ionic lipophilic surfactants.

Suitable hydrophilic surfactants include reaction products of natural or hydrogenated vegetable oils and ethylene glycol, i.e. polyoxyethylene glycolated natural or hydrogenated vegetable oils, for example polyoxyethylene glycolated natural or hydrogenated castor oils. Such products may be obtained in known manner, e.g., by reaction of a natural or hydrogenated castor oil or fractions thereof with ethylene oxide, e.g., in a molar ratio of from about 1:35 to about 1:60, with optional removal of free polyethyleneglycol components from the product, e.g., in accordance with the methods disclosed in German Auslegeschriften 1,182,388 and 1,518,819.

Suitable hydrophilic surfactants for use in the present pharmaceutical compounds also include polyoxyethylene-sorbitan-fatty acid esters, e.g., mono- and trilauryl, palmityl, stearyl and oleyl esters, e.g., of the type known and commercially available under the trade name TWEEN; including the products:

TWEEN 20 (polyoxyethylene(20)sorbitanmonolaurate),

TWEEN 40 (polyoxyethylene(20)sorbitanmonopalmitate),

TWEEN 60 (polyoxyethylene(20)sorbitanmonostearate),

TWEEN 80 (polyoxyethylene(20)sorbitanmonooleate),

TWEEN 65 (polyoxyethylene(20)sorbitantristearate),

TWEEN 85 (polyoxyethylene(20)sorbitantrioleate),

TWEEN 21 (polyoxyethylene(4)sorbitanmonolaurate),

TWEEN 61 (polyoxyethylene(4)sorbitamnonostearate), and

TWEEN 81 (polyoxyethylene(5)sorbitanmonooleate).

Especially preferred products of this class for use in the compositions of the invention are the above products TWEEN 40 and TWEEN 80. (See Hauer, et al., U.S. Pat. No. 5,342,625).

Also suitable as hydrophilic surfactants for use in the present pharmaceutical compounds are polyoxyethylene alkylethers; polyoxyethylene glycol fatty acid esters, for example polyoxythylene stearic acid esters; polyglycerol fatty acid esters; polyoxyethylene glycerides; polyoxyethylene vegetable oils; polyoxyethylene hydrogenated vegetable oils; reaction mixtures of polyols and, e.g., fatty acids, glycerides, vegetable oils, hydrogenated vegetable oils, and sterols; polyoxyethylene-polyoxypropylene co-polymers; polyoxyethylene-polyoxypropylene block co-polymers; dioctylsuccinate, dioctylsodiumsulfosuccinate, di-[2-ethylhexyl]-succinate or sodium lauryl sulfate; phospholipids, in particular lecithins such as, e.g., soya bean lecithins; propylene glycol mono- and di-fatty acid esters such as, e.g., propylene glycol dicaprylate, propylene glycol dilaurate, propylene glycol hydroxystearate, propylene glycol isostearate, propylene glycol laurate, propylene glycol ricinoleate, propylene glycol stearate, and, especially preferred, propylene glycol caprylic-capric acid diester; and bile salts, e.g., alkali metal salts, for example sodium taurocholate.

Suitable lipophilic surfactants include alcohols; polyoxyethylene alkylethers; fatty acids; bile acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid esters of mono/diglycerides; propylene glycol diglycerides; sorbitan fatty acid esters; polyoxyethylene sorbitan fatty acid esters; polyoxyethylene-polyoxypropylene block copolymers; trans-esterified vegetable oils; sterols; sugar esters; sugar ethers; sucroglycerides; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; and mixtures thereof.

Suitable lipophilic surfactants for use in the present pharmaceutical compounds also include trans-esterification products of natural vegetable oil triglycerides and polyalkylene polyols. Such trans-esterification products are known in the art and may be obtained e.g., in accordance with the general procedures described in U.S. Pat. No. 3,288,824. They include trans-esterification products of various natural (e.g., non-hydrogenated) vegetable oils for example, maize oil, kernel oil, almond oil, ground nut oil, olive oil and palm oil and mixtures thereof with polyethylene glycols, in particular polyethylene glycols having an average molecular weight of from 200 to 800. Preferred are products obtained by trans-esterification of 2 molar parts of a natural vegetable oil triglyceride with one molar part of polyethylene glycol (e.g., having an average molecular weight of from 200 to 800). Various forms of trans-esterification products of the defined class are known and commercially available under the trade name LABRAFIL.

Additional lipophilic surfactants that are suitable for use with the present pharmaceutical compositions include oil-soluble vitamin derivatives, e.g., tocopherol PEG-1000 succinate (“vitamin E TPGS”).

Also suitable as lipophilic surfactants for use in the present pharmaceutical compounds are mono-, di- and mono/di-glycerides, especially esterification products of caprylic or capric acid with glycerol; sorbitan fatty acid esters; pentaerythritol fatty acid esters and polyalkylene glycol ethers, for example pentaerythrite- -dioleate, -distearate, -monolaurate, -polyglycol ether and -monostearate as well as pentaerythrite-fatty acid esters; monoglycerides, e.g., glycerol monooleate, glycerol monopalmitate and glycerol monostearate; glycerol triacetate or (1,2,3)-triacetin; and sterols and derivatives thereof, for example cholesterols and derivatives thereof, in particular phytosterols, e.g., products comprising sitosterol, campesterol or stigmasterol, and ethylene oxide adducts thereof, for example soya sterols and derivatives thereof.

It is understood by those of ordinary skill in the art that several commercial surfactant compositions contain small to moderate amounts of triglycerides, typically as a result of incomplete reaction of a triglyceride starting material in, for example, a trans-esterification reaction. Thus, the surfactants that are suitable for use in the present pharmaceutical compositions include those surfactants that contain a triglyceride. Examples of commercial surfactant compositions containing triglycerides include some members of the surfactant families GELUCIRES, MAISINES, and IMWITORS. Specific examples of these compounds are GELUCIRE 44/14 (saturated polyglycolized glycerides); GELUCIRE 50/13 (saturated polyglycolized glycerides); GELUCIRE 53/10 (saturated polyglycolized glycerides); GELUCIRE 33/01 (semi-synthetic triglycerides of C₈-C₁₈ saturated fatty acids); GELUCIRE 39/01 (semi-synthetic glycerides); other GELUCIRES, such as 37/06, 43/01, 35/10, 37/02, 46/07, 48/09, 50/02, 62/05, etc.; MAISINE 35-I (linoleic glycerides); and IMWITOR 742 (caprylic/capric glycerides). (See U.S. Pat. No. 6,267,985).

Still other commercial surfactant compositions having significant triglyceride content are known to those skilled in the art. It should be appreciated that such compositions, which contain triglycerides as well as surfactants, may be suitable to provide all or part of the lipophilic phase component of the of the present invention, as well as all or part of the surfactants.

The relative proportion of ingredients in the compositions of the invention will, of course, vary considerably depending on the particular type of composition concerned. The relative proportions will also vary depending on the particular function of ingredients in the composition. The relative proportions will also vary depending on the particular ingredients employed and the desired physical characteristics of the product composition, e.g., in the case of a composition for topical use, whether this is to be a free flowing liquid or a paste. Determination of workable proportions in any particular instance will generally be within the capability of a person of ordinary skill in the art. All indicated proportions and relative weight ranges described below are accordingly to be understood as being indicative of preferred or individually inventive teachings only and not as limiting the invention in its broadest aspect.

The lipophilic phase component of the invention will suitably be present in an amount of from about 30% to about 90% by weight based upon the total weight of the composition. Preferably, the lipophilic phase component is present in an amount of from about 50% to about 85% by weight based upon the total weight of the composition.

The surfactant or surfactants of the invention will suitably be present in an amount of from about 1% to 50% by weight based upon the total weight of the composition. Preferably, the surfactant(s) is present in an amount of from about 5% to about 40% by weight based upon the total weight of the composition.

The amount of active vitamin D compound or a mimic thereof in compositions of the invention will of course vary, e.g., depending on the intended route of administration and to what extent other components are present. In general, however, the active vitamin D compound or a mimic thereof of the invention will suitably be present in an amount of from about 0.005% to 20% by weight based upon the total weight of the composition. Preferably, the active vitamin D compound is present in an amount of from about 0.01% to 15% by weight based upon the total weight of the composition.

The hydrophilic phase component of the invention will suitably be present in an amount of from about 2% to about 20% by weight based upon the total weight of the composition. Preferably, the hydrophilic phase component is present in an amount of from about 5% to 15% by weight based upon the total weight of the composition.

The pharmaceutical composition of the invention may be in a semisolid formulation. Semisolid formulations within the scope of the invention may comprise, e.g., a lipophilic phase component present in an amount of from about 60% to about 80% by weight based upon the total weight of the composition, a surfactant present in an amount of from about 5% to about 35% by weight based upon the total weight of the composition, and an active vitamin D compound present in an amount of from about 0.01% to about 15% by weight based upon the total weight of the composition.

The pharmaceutical compositions of the invention may be in a liquid formulation. Liquid formulations within the scope of the invention may comprise, e.g., a lipophilic phase component present in an amount of from about 50% to about 60% by weight based upon the total weight of the composition, a surfactant present in an amount of from about 4% to about 25% by weight based upon the total weight of the composition, an active vitamin D compound present in an amount of from about 0.01% to about 15% by weight based upon the total weight of the composition, and a hydrophilic phase component present in an amount of from about 5% to about 10% by weight based upon the total weight of the composition.

Additional compositions that may be used include the following, wherein the percentage of each component is by weight based upon the total weight of the composition excluding the active vitamin D compound or a mimic thereof:

| | | |
|---|---|---|
| a. | Gelucire 44/14 | about 50% |
| | Miglyol 812 | about 50%; |
| b. | Gelucire 44/14 | about 50% |
| | Vitamin E TPGS | about 10% |
| | Miglyol 812 | about 40%; |
| c. | Gelucire 44/14 | about 50% |
| | Vitamin E TPGS | about 20% |
| | Miglyol 812 | about 30%; |
| d. | Gelucire 44/14 | about 40% |
| | Vitamin E TPGS | about 30% |
| | Miglyol 812 | about 30%; |
| e. | Gelucire 44/14 | about 40% |
| | Vitamin E TPGS | about 20% |
| | Miglyol 812 | about 40%; |
| f. | Gelucire 44/14 | about 30% |
| | Vitamin E TPGS | about 30% |
| | Miglyol 812 | about 40%; |
| g. | Gelucire 44/14 | about 20% |
| | Vitamin E TPGS | about 30% |
| | Miglyol 812 | about 50%; |
| h. | Vitamin E TPGS | about 50% |
| | Miglyol 812 | about 50%; |
| i. | Gelucire 44/14 | about 60% |
| | Vitamin E TPGS | about 25% |
| | Miglyol 812 | about 15%; |
| j. | Gelucire 50/13 | about 30% |
| | Vitamin E TPGS | about 5% |
| | Miglyol 812 | about 65%; |
| k. | Gelucire 50/13 | about 50% |
| | Miglyol 812 | about 50%; |
| l. | Gelucire 50/13 | about 50% |
| | Vitamin E TPGS | about 10% |
| | Miglyol 812 | about 40%; |
| m. | Gelucire 50/13 | about 50% |
| | Vitamin E TPGS | about 20% |
| | Miglyol 812 | about 30%; |
| n. | Gelucire 50/13 | about 40% |
| | Vitamin E TPGS | about 30% |
| | Miglyol 812 | about 30%; |
| o. | Gelucire 50/13 | about 40% |
| | Vitamin E TPGS | about 20% |
| | Miglyol 812 | about 40%; |
| p. | Gelucire 50/13 | about 30% |
| | Vitamin E TPGS | about 30% |
| | Miglyol 812 | about 40%; |
| q. | Gelucire 50/13 | about 20% |
| | Vitamin E TPGS | about 30% |
| | Miglyol 812 | about 50%; |
| r. | Gelucire 50/13 | about 60% |
| | Vitamin E TPGS | about 25% |
| | Miglyol 812 | about 15%; |
| s. | Gelucire 44/14 | about 50% |
| | PEG 4000 | about 50%; |
| t. | Gelucire 50/13 | about 50% |
| | PEG 4000 | about 50%; |
| u. | Vitamin E TPGS | about 50% |
| | PEG 4000 | about 40%; |
| v. | Gelucire 44/14 | about 33.3% |
| | Vitamin E TPGS | about 33.3% |
| | PEG 4000 | about 33.3%; |
| w. | Gelucire 50/13 | about 33.3% |
| | Vitamin E TPGS | about 33.3% |
| | PEG 4000 | about 33.3%; |
| x. | Gelucire 44/14 | about 50% |
| | Vitamin E TPGS | about 50%; |
| y. | Gelucire 50/13 | about 50% |
| | Vitamin E TPGS | about 50%; |
| z. | Vitamin E TPGS | about 5% |
| | Miglyol 812 | about 95%; |
| aa. | Vitamin E TPGS | about 5% |
| | Miglyol 812 | about 65% |
| | PEG 4000 | about 30%; |
| ab. | Vitamin E TPGS | about 10% |
| | Miglyol 812 | about 90%; |
| ac. | Vitamin E TPGS | about 5% |
| | Miglyol 812 | about 85% |
| | PEG 4000 | about 10%; and |
| ad. | Vitamin E TPGS | about 10% |
| | Miglyol 812 | about 80% |
| | PEG 4000 | about 10%. |

In one embodiment of the invention, the pharmaceutical compositions comprise an active vitamin D compound or a mimic thereof, a lipophilic component, and a surfactant. The lipophilic component may be present in any percentage from about 1% to about 100%. The lipophilic component may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. The surfactant may be present in any percentage from about 1% to about 100%. The surfactant may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In one embodiment, the lipophilic component is MIGLYOL 812 and the surfactant is vitamin E TPGS. In preferred embodiments, the pharmaceutical compositions comprise about 50% MIGLYOL 812 and about 50% vitamin E TPGS, about 90% MIGLYOL 812 and about 10% vitamin E TPGS, or about 95% MIGLYOL 812 and about 5% vitamin E TPGS.

In another embodiment of the invention, the pharmaceutical compositions comprise an active vitamin D compound or a mimic thereof and a lipophilic component, e.g., around 100% MIGLYOL 812.

In a preferred embodiment, the pharmaceutical compositions comprise about 50% MIGLYOL 812, about 50% vitamin E TPGS, and small amounts of BHA and BHT. This formulation has been shown to be unexpectedly stable, both chemically and physically (see Example 3). The enhanced stability provides the compositions with a longer shelf life. Importantly, the stability also allows the compositions to be stored at room temperature, thereby avoiding the complication and cost of storage under refrigeration. Additionally, this composition is suitable for oral administration and has been shown to be capable of solubilizing high doses of active vitamin D compound, thereby enabling high dose pulse administration of active vitamin D compounds for the treatment of hyperproliferative diseases and other disorders.

In certain embodiments, the pharmaceutical compositions comprise about 50% MIGLYOL 812, about 50% vitamin E TPGS, and about 0.01% to about 0.50% each of BHA and BHT. In other embodiments, the pharmaceutical compositions comprise about 50% MIGLYOL 812, about 50% vitamin E TPGS, and about 0.05% to about 0.35% each of BHA and BHT. In certain embodiments, the pharmaceutical compositions comprise about 50% MIGLYOL 812, about 50% vitamin E TPGS, about 0.35% BHA, and about 0.10% BHT.

Additional compositions that may be used include the following, wherein the percentage of each component is by weight based upon the total weight of the composition excluding the active vitamin D compound or a mimic thereof:

| | | |
|---|---|---|
| a. | Miglyol 812 | about 100% |
| | BHA | about 0.05% |
| | BHT | about 0.05%; |
| b. | Miglyol 812 | about 100% |
| | BHA | about 0.35% |
| | BHT | about 0.10%; |
| c. | Miglyol 812 | about 50% |
| | Vitamin E TPGS | about 50% |
| | BHA | about 0.05% |
| | BHT | about 0.05%; |
| d. | Miglyol 812 | about 50% |
| | Vitamin E TPGS | about 50% |
| | BHT | about 0.10%; |
| e. | Miglyol 812 | about 50% |
| | Vitamin E TPGS | about 50% |
| | BHA | about 0.35%; |
| f. | Miglyol 812 | about 50% |
| | Vitamin E TPGS | about 50% |
| | BHA | about 0.35% |
| | BHT | about 0.10%; and |
| g. | Miglyol 812 | about 50% |
| | Vitamin E TPGS | about 50% |
| | BHA | about 0.28% |
| | BHT | about 0.08%. |

It will be understood by those of skill in the art that the formulations of the invention comprising a lipophilic component and a surfactant in amounts that total about 100% (e.g., about 50% lipophilic component and about 50% surfactant) provide adequate room for the active vitamin D compound and additives (e.g., antioxidants) which are present in the formulation in small amounts, each generally present at less than 1% by weight.

The pharmaceutical compositions comprising the active vitamin D compound or a mimic thereof of the present invention may further comprise one or more additives. Additives that are well known in the art include, e.g., detackifiers, anti-foaming agents, buffering agents, antioxidants (e.g., ascorbyl palmitate, butyl hydroxy anisole (BHA), butyl hydroxy toluene (BHT) and tocopherols, e.g., α-tocopherol (vitamin E)), preservatives, chelating agents, viscomodulators, tonicifiers, flavorants, colorants odorants, opacifiers, suspending agents, binders, fillers, plasticizers, lubricants, and mixtures thereof. The amounts of such additives can be readily determined by one skilled in the art, according to the particular properties desired. For example, antioxidants such as BHA and BHT may each be present in an amount of from about 0.01% to about 0.50% by weight based upon the total weight of the composition, e.g., about 0.05 to about 0.35% by weight, e.g., about 0.01, 0.05, 0.10, 0.15, 0.20, 0.25, 0.30, 0.35, 0.40, 0.45, or 0.50% by weight.

The additive may also comprise a thickening agent. Suitable thickening agents may be of those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g., hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropylmethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products.

Such thickening agents as described above may be included, e.g., to provide a sustained release effect. However, where oral administration is intended, the use of thickening agents as aforesaid will generally not be required and is generally less preferred. Use of thickening agents is, on the other hand, indicated, e.g., where topical application is foreseen.

Compositions in accordance with the present invention may be employed for administration in any appropriate manner, e.g., orally, e.g., in unit dosage form, for example in a solution, in hard or soft encapsulated form including gelatin encapsulated form, parenterally or topically, e.g., for application to the skin, for example in the form of a cream, paste, lotion, gel, ointment, poultice, cataplasm, plaster, dermal patch or the like, or for ophthalmic application, for example in the form of an eye-drop, -lotion or -gel formulation. Readily flowable forms, for example solutions and emulsions, may also be employed e.g., for intralesional injection, or may be administered rectally, e.g., as an enema.

When the composition of the present invention is formulated in unit dosage form, the active vitamin D compound or a mimic thereof will preferably be present in an amount of between 1 and 1000 μg per unit dose. More preferably, the amount of active vitamin D compound per unit dose will be about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, 350, 400, 450, 500, 550, 600, 650, 700, 750, 800, 850, 900, 950, or 1000 μg or any amount therein. In one embodiment, the amount of active vitamin D compound per unit dose will be about 5 μg to about 180 μg, more preferably about 10 μg to about 135 μg, more preferably about 45 μg. In one embodiment, the unit dosage form comprises 45, 90, 135, or 180 μg of calcitriol.

When the unit dosage form of the composition is a capsule, the total quantity of ingredients present in the capsule is preferably about 10-1000 μL. More preferably, the total quantity of ingredients present in the capsule is about 100-300 μL. In another embodiment, the total quantity of ingredients present in the capsule is preferably about 10-1500 mg, preferably about 100-1000 mg. In one embodiment, the total quantity is about 225, 450, 675, or 900 mg. In one embodiment, the unit dosage form is a capsule comprising 45, 90, 135, or 180 μg of calcitriol.

Therapeutic Agents Used in Combination with Active Vitamin D Compounds

One aspect of the present invention provides methods for treating, ameliorating, preventing an immune-mediated disorder comprising administering to an animal in need thereof an active vitamin D compound or a mimic thereof in combination with one or more therapeutic agents. Therapeutic agents include, but are not limited to, small molecules, synthetic drugs, peptides, polypeptides, proteins, nucleic acids (e.g., DNA and RNA nucleotides including, but not limited to, antisense nucleotide sequences, triple helices and nucleotide sequences encoding biologically active proteins, polypeptides or peptides) antibodies, synthetic or natural inorganic molecules, mimetic agents, synthetic or natural organic molecules, and small molecules. Any agent which is known to be useful, or which has been used or is currently being used for the treatment, amelioration, or prevention of one or more symptoms associated with an immune-mediated disorder can be used in combination with an active vitamin D compound or a mimic thereof in accordance with the invention described herein. See, e.g., Hardman, et al., eds., Goodman & Gilman's The Pharmacological Basis Of Therapeutics, 10th Ed, Mc-Graw-Hill, New York (1996), at pages 643-754, 1381-1484, 1649-1678, and the emedicine website (www.emedicine.com) for information regarding therapeutic agents which have been or are currently being used for treating immune-mediated disorders. Examples of such agents include, but are not limited to, immunomodulatory agents (e.g., small organic molecules, T cell receptor modulators, cytokine receptor modulators, T-cell depleting agents, cytokine antagonists, monokine antagonists, lymphocyte inhibitors, anti-cancer agents, corticosteroids, cytotoxic agents, and immunosuppressive agents); anti-angiogenic agents such as angiostatin, TNF-α antagonists (e.g., anti-TNF-α antibodies), integrin αᵥβ₃ antagonists (e.g., proteinaceous agents such as non-catalytic metalloproteinase fragments, RGD peptides, peptide mimetics, fusion proteins, disintegrins or derivatives or analogs thereof, and antibodies that immunospecifically bind to integrin αᵥβ₃, nucleic acid molecules, organic molecules, and inorganic molecules); anti-inflammatory agents (e.g., nonsteroidal anti-inflammatory agents, non-narcotic analgesics such as acetaminophen and phenacetin, chloroquine, gold salts, methotrexate, D-penicillamine, allopurinol, colchicine, probenecid, sulfinpyrazone, antihistamine agents, anti-malarial agents such as hydroxychloroquine, anti-viral agents, antibiotics, and PPARγ agonists); and dermatological agents for rashes and swellings (e.g., phototherapy, photochemotherapy, and topical agents such as emollients, salicylic acid, coal tar, topical steroids, topical corticosteroids, tazarotene, and topical retinoids). In certain embodiments, therapeutic agents include systemically administered agents such as, e.g., corticosteroids, azathioprine, methotrexate, cyclophosphamide (Cytoxan), chlorambucil, mycophenolate mofetil (CellCept), mercaptopurine, rapamune, tacrolimus (FK506), cyclosporine, retinoids, nitrogen mustard, interferon, antibiotics, antihistamines, PUVA, chemotherapy, and UV light.

Immunomodulatory Agents

Any immunomodulatory agent known to one of skill in the art may be used in the methods and compositions of the invention. An immunomodulatory agent is an agent that modulates a host's immune system. In particular, an immunomodulatory agent is an agent that alters the ability of a subject's immune system to respond to one or more foreign antigens. In a specific embodiment, an immunomodulatory agent is an agent that shifts one aspect of a subject's immune response, e.g., the agent shifts the immune response from a Th1 to a Th2 response. In certain embodiments, an immunomodulatory agent is an agent that inhibits or reduces a subject's immune system (i.e., an immunosuppressant agent). In certain other embodiments, an immunomodulatory agent is an agent that activates or increases a subject's immune system (i.e., an immunostimulatory agent). In accordance with the invention, an immunomodulatory agent used in the combination therapies of the invention does not include a vitamin D derivative or analog.

Immunomodulatory agents can affect one or more or all aspects of the immune response in a subject. Aspects of the immune response include, but are not limited to, the inflammatory response, the complement cascade, leukocyte and lymphocyte differentiation, proliferation, and/or effector function, monocyte and/or basophil counts, and the cellular communication among cells of the immune system. In certain embodiments of the invention, an immunomodulatory agent modulates one aspect of the immune response. In other embodiments, an immunomodulatory agent modulates more than one aspect of the immune response. In a preferred embodiment of the invention, the administration of an immunomodulatory agent to an animal inhibits or reduces one or more aspects of the animal's immune response capabilities. In a specific embodiment of the invention, the immunomodulatory agent inhibits or suppresses the immune response in an animal. In accordance with the invention, an immunomodulatory agent is not an active vitamin D compound, e.g., calcitriol.

An immunomodulatory agent may be selected to interfere with the function of T cells and/or B cells. An immunomodulatory agent may also be selected to interfere with the interactions between T cells and B cells, e.g., interactions between the T helper subsets (TH1 or TH2) and B cells to inhibit neutralizing antibody formation. An immunomodulatory agent may be selected to inhibit the interaction between TH1 cells and cytotoxic lymphocytes (CTLs) to reduce the occurrence of CTL-mediated killing. An immunomodulatory agent may be selected to alter (e.g., inhibit or suppress) the proliferation, differentiation, activity and/or function of CD4⁺ and/or CD8⁺ T cells. For example, antibodies specific for T cells can be used as immunomodulatory agents to deplete, or alter the proliferation, differentiation, activity and/or function of CD4⁺ and/or CD8⁺ T cells. Examples of immunomodulatory agents include, but are not limited to, proteinaceous agents such as cytokines, peptide mimetics, and antibodies (e.g., human antibodies, humanized antibodies, camelised antibodies, chimeric antibodies, monoclonal antibodies, polyclonal antibodies, single domain antibodies, Fvs, ScFvs, Fab or F(ab)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide (Cytoxan), azathioprine (Immuran), cyclosporine, minocycline, antibiotics, tacrolimus (FK506), methylprednisolone, corticosteroids, steroids, mycophenolate mofetil (CellCept), rapamycin (sirolimus), chlorambucil, mizoribine, deoxyspergualin, brequinar, malononitriloamides, T cell modulators, B cell modulators, and cytokine receptor modulators. Examples of T cell modulators include, but are not limited to, anti-T cell receptor antibodies (e.g., anti-CD4 antibodies (e.g., cM-T412 (Boeringer), IDEC-CE9.1 (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), and anti-B7 antibodies (e.g., IDEC-114) (IDEC))) and CTLA4-immunoglobulin (CTLA4-Ig). Examples of B cell modulators include, but are not limited to, anti-B cell receptor antibodies, anti-CD19 antibodies, and anti-CD20 antibodies (e.g., Rituxan (IDEC), Bexxar). Examples of cytokine receptor modulators include, but are not limited to, soluble cytokine receptors (e.g., the extracellular domain of a TNF-α receptor or a fragment thereof, the extracellular domain of an IL-1 receptor or a fragment thereof, and the extracellular domain of an IL-6 receptor or a fragment thereof), cytokines or fragments thereof (e.g., IL-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1 antibodies, anti-IL-6 antibodies, anti-IL-8 antibodies (e.g., ABX-IL-8 (Abgenix)), anti-IL-9 antibodies and anti-IL-12 antibodies. In a specific embodiment, a cytokine receptor modulator is IL-4, IL-10, or a fragment thereof. In another embodiment, a cytokine receptor modulator is an anti-IL-1 antibody, anti-IL-6 antibody, anti-IL-12 receptor antibody, or anti-TNF-α antibody. In another embodiment, a cytokine receptor modulator is the extracellular domain of a TNF-α receptor or a fragment thereof In a preferred embodiment, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the animal is a human, the proteins, polypeptides, or peptides that are utilized as immunomodulatory agents are human or humanized.

In accordance with the invention, one or more immunomodulatory agents are administered to an animal with an immune-mediated disorder prior to, subsequent to, or concomitantly with the active vitamin D compound or a mimic thereof and other therapeutic agents of the invention. Preferably, one or more immunomodulatory agents are administered to an animal with an immune-mediated disorder to reduce or inhibit one or more aspects of the immune response as necessary. Any technique well-known to one skilled in the art can be used to measure one or more aspects of the immune response in a particular animal, and thereby determine when it is necessary to administer an immunomodulatory agent to said animal. In a preferred embodiment, a mean absolute lymphocyte count of approximately 500 cells/mm³, preferably 600 cells/mm³, 650 cells/mm³, 700 cells/mm³, 750 cells/mm³, 800 cells/mm³, 900 cells/mm³, 1000 cells/mm³, 1100 cells/mm³, or 1200 cells/mm³ is maintained in a subject. In another preferred embodiment, an animal with an immune-mediated disorder is not administered an immunomodulatory agent if their absolute lymphocyte count is 500 cells/mm³ or less, 550 cells/mm³ or less, 600 cells/mm³ or less, 650 cells/mm³ or less, 700 cells/mm³ or less, 750 cells/mm³ or less, or 800 cells/mm³ or less.

In a preferred embodiment, one or more immunomodulatory agents are administered to an animal with an immune-mediated disorder so as to transiently reduce or inhibit one or more aspects of the immune response. Such a transient inhibition or reduction of one or more aspects of the immune system can last for hours, days, weeks, or months. Preferably, the transient inhibition or reduction in one or more aspects of the immune response lasts for a few hours (e.g., 2 hours, 4 hours, 6 hours, 8 hours, 12 hours, 14 hours, 16 hours, 18 hours, 24 hours, 36 hours, or 48 hours), a few days (e.g., 3 days, 4 days, 5 days, 6 days, 7 days, or 14 days), or a few weeks (e.g., 3 weeks, 4 weeks, 5 weeks or 6 weeks).

In one embodiment of the invention, an immunomodulatory agent that reduces or depletes T cells, preferably memory T cells, is administered to an animal with an immune-mediated disorder in accordance with the methods of the invention. In another embodiment of the invention, an immunomodulatory agent that inactivates CD8⁺ T cells is administered to an animal with an immune-mediated disease in accordance with the methods of the invention. In a specific embodiment, anti-CD8 antibodies are used to reduce or deplete CD8⁺ T cells. Antibodies that interfere with or block the interactions necessary for the activation of B cells by TH cells, and thus block the production of neutralizing antibodies, are useful as immunomodulatory agents in the methods of the invention. For example, B cell activation by T cells requires certain interactions to occur (Durie et al, Immunol. Today, 15(9):406-410 (1994)), such as the binding of CD40 ligand on the T helper cell to the CD40 antigen on the B cell, and the binding of the CD28 and/or CTLA4 ligands on the T cell to the B7 antigen on the B cell. Without both interactions, the B cell cannot be activated to induce production of the neutralizing antibody. The CD40 ligand (CD40L)-CD40 interaction is a desirable point to block the immune response because of its broad activity in both T helper cell activation and function as well as the absence of redundancy in its signaling pathway. Thus, in a specific embodiment of the invention, the interaction of CD40L with CD40 is transiently blocked at the time of administration of one or more of the immunomodulatory agents. This can be accomplished by treating with an agent which blocks the CD40 ligand on the TH cell and interferes with the normal binding of CD40 ligand on the TH cell with the CD40 antigen on the B cell. An antibody to CD40 ligand (anti-CD40L) (available from Bristol-Myers Squibb Co; see, e.g., European patent application 555,880, published Aug. 18, 1993) or a soluble CD40 molecule can be selected and used as an immunomodulatory agent in accordance with the methods of the invention. In another embodiment, an immunomodulatory agent which reduces or inhibits one or more biological activities (e.g., the differentiation, proliferation, and/or effector functions) of TH0, TH1, and/or TH2 subsets of CD4⁺ T helper cells is administered to an animal with an immune-mediated disorder in accordance with the methods of the invention. One example of such an immunomodulatory agent is IL-4. IL-4 enhances antigen-specific activity of TH2 cells at the expense of the TH1 cell function (see, e.g., Yokota, et al., Proc. Natl. Acad. Sci., USA, 83:5894-5898 (1986); and U.S. Pat. No. 5,017,691). Other examples of immunomodulatory agents that affect the biological activity (e.g., proliferation, differentiation, and/or effector functions) of T-helper cells (in particular, TH1 and/or TH2 cells) include, but are not limited to, IL-2, IL-6, IL-9, IL-10, IL-12, IL-15 and IFN-γ. In another embodiment, an immunomodulatory agent administered to an animal with an immune-mediated disorder in accordance with the methods of the invention is a cytokine that prevents antigen presentation. In a preferred embodiment, an immunomodulatory agent used in the methods of the invention is IL-10. IL-10 also reduces or inhibits macrophage action which involves bacterial elimination. Other examples of immunomodulatory agents which can be used in accordance with the invention include, but are not limited to, corticosteroids (e.g., beclomethasone, betamethasone, cortisone, desoxycorticosterone, dexamethasone, fludrocortisone, hydrocortisone, methylprednisolone, paramethasone, prednisolone, prednisone, and triamcinolone), azathioprine, mycophenolate mofetil, cyclosporin A, FK506, methotrexate, 5-fluoruracil, 6-thioguanine, cytarabine, melphalan, busulfan, carmustine, lomustine, procarbazine, decarbazine, cisplatin, carboplatin, leflunomide, and cyclophosphamide. A short course of cyclophosphamide has been demonstrated to successfully interrupt both CD4⁺ and CD8⁺ T cell activation to adenoviral capsid protein (Jooss et al., Hum. Gene Ther. 7:1555-1566 (1996)), and at higher doses, formation of neutralizing antibody was prevented. Hydrocortisone or cyclosporin A treatment has been successfully used to decrease the induction of cytokines, some of which may be involved in the clearance of bacterial infections. Nucleic acid molecules encoding proteins, polypeptides, or peptides with immunomodulatory activity or proteins, polypeptides, or peptides with immunomodulatory activity can be administered to an animal with an immune-mediated disorder in accordance with the methods of the invention. Further, nucleic acid molecules encoding derivatives, analogs, fragments or variants of proteins, polypeptides, or peptides with immunomodulatory activity, or derivatives, analogs, fragments or variants of proteins, polypeptides, or peptides with immunomodulatory activity can be administered to an animal with an immune-mediated disorder in accordance with the methods of the invention. Preferably, such derivatives, analogs, variants and fragments retain the immunomodulatory activity of the full-length wild-type protein, polypeptide, or peptide. Proteins, polypeptides, or peptides that can be used as immunomodulatory agents can be produced by any technique well-known in the art. See, e.g., Chapter 16 Ausubel et al., eds., Short Protocols in Molecular Biology, Fourth Edition, John Wiley & Sons, NY (1999), which describes methods of producing proteins, polypeptides, or peptides, and which is incorporated herein by reference in its entirety. Antibodies which can be used as immunomodulatory agents can be produced by, e.g., methods described in U.S. Pat. No. 6,245,527 and in Harlow et al., Antibodies: A Laboratory Manual, Cold Spring Harbor Laboratory Press, Cold Spring Harbor, N.Y. (1988), which are incorporated herein by reference in their entirety. Preferably, agents that are commercially available and known to function as immunomodulatory agents are used in the compositions and methods of the invention. The immunomodulatory activity of an agent can be determined in vitro and/or in vivo by any technique well-known to one skilled in the art, including, e.g., by CTL assays, proliferation assays, and immunoassays (e.g. ELISAs) for the expression of particular proteins such as co-stimulatory molecules and cytokines.

Anti-angiogenic Agents

Any anti-angiogenic agents well-known to one of skill in the art can be used in the compositions and methods of the invention. Non-limiting examples of anti-angiogenic agents include proteins, polypeptides, peptides, fusion proteins, antibodies (e.g., human antibodies, humanized antibodies, camelised antibodies, chimeric antibodies, monoclonal antibodies, polyclonal antibodies, single domain antibodies, Fvs, ScFvs, Fab fragments, F(ab)₂ fragments, and antigen-binding fragments thereof), nucleic acid molecules (e.g., antisense molecules or triple helices), organic molecules, inorganic molecules, and small molecules that reduce or inhibit or neutralize the angiogenesis. In particular, examples of anti-angiogenic agents include, but are not limited to, endostatin, angiostatin, apomigren, anti-angiogenic antithrombin III, the 29 kDa N-terminal and 40 kDa C-terminal proteolytic fragments of fibronectin, a uPA receptor antagonist, the 16 kDa proteolytic fragment of prolactin, the 7.8 kDa proteolytic fragment of platelet factor-4, the anti-angiogenic 24 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, RGD and NGR containing peptides, the small anti-angiogenic peptides of laminin, fibronectin, procollagen and EGF, integrin αᵥβ₃ antagonists (e.g., anti-integrin αᵥβ₃ antibodies), TNF-α antagonists, acid fibroblast growth factor (aFGF) antagonists, basic fibroblast growth factor (bFGF) antagonists, vascular endothelial growth factor (VEGF) antagonists, and VEGF receptor (VEGFR) antagonists (e.g., anti-VEGFR antibodies).

In a specific embodiment of the invention, an anti-angiogenic agent is endostatin. Naturally occurring endostatin consists of the C-terminal ˜180 amino acids of collagen XVIII (cDNAs encoding two splice forms of collagen XVIII have GenBank Accession Nos. AF18081 and AF18082). In another embodiment of the invention, an anti-angiogenic agent is a plasminogen fragment (the coding sequence for plasminogen can be found in GenBank Accession Nos. NM_{—}000301 and A33096). Angiostatin peptides naturally include the four kringle domains of plasminogen, kringle 1 through kringle 4. It has been demonstrated that recombinant kringle 1, 2 and 3 possess the anti-angiogenic properties of the native peptide, whereas kringle 4 has no such activity (Cao et al., J. Biol. Chem. 271:29461-29467 (1996)). Accordingly, the angiostatin peptides comprise at least one and preferably more than one kringle domain selected from the group consisting of kringle 1, kringle 2 and kringle 3. In a specific embodiment, the anti-angiogenic peptide is the 40 kDa isoform of the human angiostatin molecule, the 42 kDa isoform of the human angiostatin molecule, the 45 kDa isoform of the human angiostatin molecule, or a combination thereof. In another embodiment, an anti-angiogenic agent is the kringle 5 domain of plasminogen, which is a more potent inhibitor of angiogenesis than angiostatin (angiostatin comprises kringle domains 1-4). In another embodiment of the invention, an anti-angiogenic agent is antithrombin III. Antithrombin III, which is referred to hereinafter as antithrombin, comprises a heparin binding domain that tethers the protein to the vasculature walls, and an active site loop which interacts with thrombin. When antithrombin is tethered to heparin, the protein elicits a conformational change that allows the active loop to interact with thrombin, resulting in the proteolytic cleavage of said loop by thrombin. The proteolytic cleavage event results in another change of conformation of antithrombin, which (i) alters the interaction interface between thrombin and antithrombin and (ii) releases the complex from heparin (Carrell, Science 285:1861-1862 (1999), and references therein). O'Reilly et al., Science 285:1926-1928 (1999), have discovered that the cleaved antithrombin has potent anti-angiogenic activity. Accordingly, in one embodiment, an anti-angiogenic agent is the anti-angiogenic form of antithrombin. In another embodiment of the invention, an anti-angiogenic agent is the 40 kDa and/or 29 kDa proteolytic fragment of fibronectin.

In another embodiment of the invention, an anti-angiogenic agent is a urokinase plasminogen activator (uPA) receptor antagonist. In one mode of the embodiment, the antagonist is a dominant negative mutant of uPA (see, e.g., Crowley et al., Proc. Natl. Acad. Sci. USA 90:5021-5025 (1993)). In another mode of the embodiment, the antagonist is a peptide antagonist or a fusion protein thereof (Goodson et al., Proc. Natl. Acad. Sci. USA 91:7129-7133 (1994)). In yet another mode of the embodiment, the antagonist is a dominant negative soluble uPA receptor (Min et al., Cancer Res. 56:2428-2433 (1996)). In another embodiment of the invention, a therapeutic molecule of the invention is the 16 kDa N-terminal fragment of prolactin, comprising approximately 120 amino acids, or a biologically active fragment thereof (the coding sequence for prolactin can be found in GenBank Accession No. NM_{—}000948). In another embodiment of the invention, an anti-angiogenic agent is the 7.8 kDa platelet factor-4 fragment. In another embodiment of the invention, a therapeutic molecule of the invention is a small peptide corresponding to the anti-angiogenic 13 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, the small anti-angiogenic peptides of laminin, fibronectin, procollagen, or EGF, or small peptide antagonists of integrin αᵥβ₃ or the VEGF receptor. In another embodiment, the small peptide comprises an RGD or NGR motif. In certain embodiments, an anti-angiogenic agent is a TNF-α antagonist.

TNF-α Antagonists

Any TNF-α antagonist well-known to one of skill in the art can be used in the compositions and methods of the invention. Non-limiting examples of TNF-α antagonists include proteins, polypeptides, peptides, fusion proteins, antibodies (e.g., human antibodies, humanized antibodies, camelised antibodies, chimeric antibodies, monoclonal antibodies, polyclonal antibodies, single domain antibodies, Fvs, ScFvs, Fab fragments, F(ab)₂ fragments, and antigen-binding fragments thereof), nucleic acid molecules (e.g., antisense molecules or triple helices), organic molecules, inorganic molecules, and small molecules that block, reduce, inhibit or neutralize a function, an activity and/or expression of TNF-α. In various embodiments, a TNF-α antagonist reduces the function, activity and/or expression of TNF-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

Examples of antibodies that immunospecifically bind to TNF-α include, but are not limited to, infliximab (REMICADE™; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., Proc. Natl. Acad. Sci. USA 91: 2762-2766 (1994); Thorbecke et al., Proc. Natl. Acad. Sci. USA 89:7375-7379 (1992)). The present invention also encompasses the use of antibodies that immunospecifically bind to TNF-α disclosed in the following U.S. patents in the compositions and methods of the invention: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety. Examples of soluble TNF-α receptors include, but are not limited to, sTNF-R1 (Amgen), etanercept (ENBREL™; Immunex) and its rat homolog RENBREL™, soluble inhibitors of TNF-α derived from TNFrI, TNFrII (Kohno et al., Proc. Natl. Acad. Sci. USA 87:8331-8335 (1990), and TNF-α lnh (Seckinger et al, Proc. Natl. Acad. Sci. USA 87:5188-5192 (1990)).

In one embodiment, a TNF-α antagonist used in the methods of the invention is a soluble TNF-α receptor. In a specific embodiment, a TNF-α antagonist used in the compositions and methods of the invention is etanercept (ENBREL™; Immunex) or a fragment, derivative or analog thereof. In another embodiment, a TNF-α antagonist used in the compositions and methods of the invention is an antibody that immunospecifically binds to TNF-α. In a specific embodiment, a TNF-α antagonist used in the compositions and methods of the invention is infliximab (REMICADE™; Centacor) or a derivative, analog or antigen-binding fragment thereof. Other TNF-α antagonists encompassed by the invention include, but are not limited to, IL-10, which is known to block TNF-α production via interferon-activated macrophages (Oswald et al., Proc. Natl. Acad. Sci. USA 89:8676-8680 (1992)), TNFR-IgG (Ashkenazi et al., Proc. Natl. Acad. Sci. USA 88:10535-10539 (1991)), the murine product TBP-1 (Serono/Yeda), the vaccine CytoTAb (Protherics), antisense molecule104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SCIO-469 (Scios), TACE targeter (Immunix/AFHP), CLX-120500 (Calyx), Thiazolopyrim (Dynavax), auranofin (Ridaura; SmithKline Beecham Pharmaceuticals), quinacrine (mepacrine dichlorohydrate), tenidap (Enablex), melanin (Large Scale Biological), and anti-p38 MAPK agents by Uriach. Nucleic acid molecules encoding proteins, polypeptides, or peptides with TNF-α antagonist activity or proteins, polypeptides, or peptides with TNF-α antagonist activity can be administered to an animal with an immune-mediated disorder in accordance with the methods of the invention. Further, nucleic acid molecules encoding derivatives, analogs, fragments or variants of proteins, polypeptides, or peptides with TNF-α antagonist activity, or derivatives, analogs, fragments or variants of proteins, polypeptides, or peptides with TNF-α antagonist activity can be administered to an animal with an immune-mediated disorder in accordance with the methods of the invention. Preferably, such derivatives, analogs, variants and fragments retain the TNF-α antagonist activity of the full-length wild-type protein, polypeptide, or peptide. Proteins, polypeptides, or peptides that can be used as TNF-α antagonists can be produced by any technique well-known in the art. Proteins, polypeptides or peptides with TNF-α antagonist activity can be engineered so as to increase the in vivo half-life of such proteins, polypeptides, or peptides utilizing techniques well-known in the art. Preferably, agents that are commercially available and known to function as TNF-α antagonists are used in the compositions and methods of the invention. The TNF-α antagonist activity of an agent can be determined in vitro and/or in vivo by any technique well-known to one skilled in the art.

Integrin αᵥβ₃ Antagonists

Any integrin αᵥβ₃ antagonist well-known to one of skill in the art may be used in the methods and compositions of the invention. As used herein, the “integrin αᵥβ₃ antagonist” and analogous terms refer to any protein, polypeptide, peptide, fusion protein, antibody, antibody fragment, large molecule, or small molecule that blocks, inhibits, reduces, or neutralizes the function, activity, and/or expression of integrin αᵥβ₃. In various embodiments, an integrin αᵥβ₃ antagonist reduces the function, activity and/or expression of integrin αᵥβ₃ by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as PBS. The invention encompasses the use of one or more integrin αᵥβ₃ antagonists in the compositions and methods of the invention. Examples of integrin αᵥβ₃ antagonists include, but are not limited to, proteinaceous agents such as non-catalytic metalloproteinase fragments, RGD peptides, peptide mimetics, fusion proteins, disintegrins or derivatives or analogs thereof, and antibodies that immunospecifically bind to integrin αᵥβ₃, nucleic acid molecules, organic molecules, and inorganic molecules. Non-limiting examples of RGD peptides recognized by integrin αᵥβ₃ include Triflavin. Examples of antibodies that immunospecifically bind to integrin αᵥβ₃ include, but are not limited to, 11D2 (Searle) and LM609 (Scripps). Non-limiting examples of small molecule peptidomimetic integrin αᵥβ₃ antagonists include S836 (Searle) and S448 (Searle). Examples of disintegrins include, but are not limited to, Accutin. The invention also encompasses the use of any of the integrin αᵥβ₃ antagonists disclosed in the following U.S. patents in the methods of the invention: U.S. Pat. Nos. 5,149,780; 5,196,511; 5,204,445; 5,262,520; 5,306,620; 5,478,725; 5,498,694; 5,523,209; 5,578,704; 5,589,570; 5,652,109; 5,652,110; 5,693,612; 5,705,481; 5,767,071; 5,770,565; 5,780,426; 5,817,457; 5,830,678; 5,849,692; 5,955,572; 5,985,278; 6,048,861; 6,090,944; 6,096,707; 6,130,231; 6,153,628; 6,160,099; and 6,171,588, each of which is incorporated herein by reference in its entirety. In certain embodiments, an integrin Cαᵥβ₃ antagonist is a small organic molecule. In a preferred embodiment, an integrin αᵥβ₃ antagonist is an antibody that immunospecifically binds to integrin αᵥβ₃. In a preferred embodiment, integrin αᵥβ₃ antagonists inhibit or reduce angiogenesis.

In a preferred embodiment, proteins, polypeptides or peptides (including antibodies and fusion proteins) that are utilized as integrin αᵥβ₃ antagonists are derived from the same species as the recipient of the proteins, polypeptides or peptides so as to reduce the likelihood of an immune response to those proteins, polypeptides or peptides. In another preferred embodiment, when the animal is a human, the proteins, polypeptides, or peptides that are utilized as integrin αᵥβ₃ antagonists are human or humanized.

In accordance with the invention, one or more integrin αᵥβ₃ antagonists are administered to an animal with an immune-mediated disorder prior to, subsequent to, or concomitantly with an active vitamin D compound and one or more other therapeutic agents which have been used, are currently being used or are known to be useful in the treatment of said immune-mediated disorder. Nucleic acid molecules encoding proteins, polypeptides, or peptides that function as integrin αᵥβ₃ antagonists, or proteins, polypeptides, or peptides that function as integrin αᵥβ₃ antagonists can be administered to an animal with an immune-mediated disorder in accordance with the methods of the invention. Further, nucleic acid molecules encoding derivatives, analogs, fragments or variants of proteins, polypeptides, or peptides that function as integrin αᵥβ₃ antagonists, or derivatives, analogs, fragments or variants of proteins, polypeptides, or peptides that function as integrin αᵥβ₃ antagonists can be administered to a subject with an immune-mediated disorder in accordance with the methods of the invention. Preferably, such derivatives, analogs, variants and fragments retain the integrin αᵥβ₃ antagonist activity of the full-length wild-type protein, polypeptide, or peptide.

Anti-inflammatory Agents

Anti-inflammatory agents have exhibited success in treatment of inflammatory and autoimmune disorders and are now a common and a standard treatment for such disorders. Any anti-inflammatory agent well-known to one of skill in the art can be used in the compositions and methods of the invention. Non-limiting examples of anti-inflammatory agents include nonsteroidal anti-inflammatory agents (NSAIDs), non-narcotic analgesics such as acetaminophen and phenacetin, steroidal anti-inflammatory drugs, beta-agonists, anticholingeric agents, methyl xanthines, chloroquine, gold salts, methotrexate, D-penicillamine, allopurinol, coichicine, probenecid, sulfinpyrazone, antihistamine agents, anti-malarial agents such as hydroxychloroquine, anti-viral agents, antibiotics, and PPARγ agonists. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxygenase enzyme. Examples of steroidal anti-inflammatory drugs include, but are not limited to, glucocorticoids, dexamethasone (DECADRON™), cortisone, hydrocortisone, prednisone (DELTASONE™), prednisolone, and triamcinolone. Antihistamine agents include, but are not limited to, alkylamines (e.g., brompheniramine, chlorpheniramine, dexchlorpheniramine, and triprolidine), ethanolamines (e.g., carbinoxamine, clemastine, dimenhydrinate, diphenhydramine, and doxylamine), ethylenediamines (e.g., tripelennamine and pyrilamine), phenothiazines (e.g., methdilazine, promethazine, and timeprazine), piperazines (e.g., cyclizine, hydroxyzine, and meclizine), piperidines (e.g., azatadine and cyproheptadine), terfenadine, astemazole, loratidine, and cetirizine. Anti-viral agents include, but are not limited to, amantadine, ribavirin, rimantadine, acyclovir, famciclovir, foscamet, ganciclovir, trifluridine, vidarabine, didanosine, stavudine, zalciltabine, zidovudine, and interferon. Antibiotics include, but are not limited to, antibiotics used in cancer therapy (e.g., dactinomycin, doxorubicin, daunorubicin, bleomycin, and plicamycin), inhibitors of metabolism (e.g., sulfonamides and trimethoprim), inhibitors of cell wall synthesis (e.g., β-lactams and vancomycin), inhibitors of protein synthesis (e.g., tetracyclines, aminoglycosides, macrolides, clindamycin, and chloramphenicol), and inhibitors of nucleic acid function or synthesis (e.g., fluoroquinolones and rifampin). PPARγ agonists include thiazolidinediones such as troglitazone, ciglitazone, pioglitazone, and rosiglitazone. See U.S. Pat. Nos. 5,594,015, 5,478,852, and 5,326,770.

Dermatological Agents

Any dermatological agent well-known to one of skill in the art can be used in the methods of the invention. A dermatological agent refers to an agent that helps treat skin diseases and complaints. Preferably, a dermatological agent refers to a topical agent used to prevent, treat or ameliorate a skin condition, in particular a skin condition associated with increased T cell infiltration, increased T cell activation, and/or abnormal antigen presentation. In a particularly preferred embodiment, a dermatological agent refers to a topical agent used to prevent, treat or ameliorate psoriasis or one or more symptoms thereof. Examples of dermatological agents include, but are not limited to, proteins, polypeptides, peptides, fusion proteins, antibodies (e.g., human antibodies, humanized antibodies, camelised antibodies, chimeric antibodies, monoclonal antibodies, polyclonal antibodies, single domain antibodies, Fvs, ScFvs, Fab fragments, F(ab)₂ fragments, and antigen-binding fragments thereof), nucleic acid molecules (e.g., antisense molecules or triple helices), organic molecules, inorganic molecules, and small molecules which are used to treat or ameliorate a skin condition. In a specific embodiment, the dermatological agent is phototherapy (i.e., ultraviolet B radiation) or photochemotherapy (e.g., PUVA). In accordance with the invention, a dermatological agent is not a vitamin D compound or a mimic thereof.

In a preferred embodiment, a dermatological agent is a topical agent. Examples of topical agents include, but are not limited to emollients, salicylic acid, coal tar, anthralins, topical steroids, topical corticosteroids (e.g., difloroasone diacetate, clobetasol propionate, halobetasol propionate, betamethasone dipropionate, fluocinonide, halcinonide desoximetasone, triamcinolone, fluticasone propionate, fluocinolone acetonide, flurandrenolide, mometasone furoate, betamethasone, aclometasome dipropionate, desonide, and hydrocortisone), and topical retinoids (e.g., tazarotene). In certain embodiments, a dermatological agent is a systemically administered agent. Examples of dermatological agents administered systemically include, but are not limited to, systemic corticosteroids (e.g., triamcinolone), folic acid antagonists (e.g., methotrexate), retinoids (e.g., acetretin) and cyclosporine.

Combination Therapy

The combination therapies of the invention comprise an active vitamin D compound or a mimic thereof and at least one other therapeutic agent which has a different mechanism of action than the active vitamin D compound or a mimic thereof. The mechanisms of therapeutic agents other than active vitamin D compounds which can be used in the combination therapies of the present invention can be found in the art (see, e.g., Hardman et al., eds., Goodman & Gilman's The Pharmacological Basis Of Basis Of Therapeutics 10th Ed, Mc-Graw-Hill, New York at pages 643-754, 1381-1484, 1649-1678, 1996; Physician's Desk Reference (PDR) 55th Ed., Medical Economics Co., Inc., Montvale, N.J. (2001) (www.pdr.net), and the emedicine website. The combination therapies of the present invention also comprise an active vitamin D compound or a mimic thereof and at least one other therapeutic agent which improves the therapeutic effect of the active vitamin D compound or a mimic thereof by functioning together with the active vitamin D compound or a mimic thereof to have an additive or synergistic effect. In accordance with the present invention, at least one active vitamin D compound or a mimic thereof and one other therapeutic agent that functions differently from the active vitamin D compound or a mimic thereof are advantageously utilized in combination for the treatment or amelioration of an immune-mediated disorder. An active vitamin D compound or a mimic thereof may be administered prior to (e.g., 0.5 hours, 1 hour, 2 hours, 4 hours, 6 hours, 12 hours, 24 hours, 36 hours, 48 hours, 5 days, 1 week, 2 weeks, 1 month or more before), subsequent to (e.g., 0.5 hours, 1 hour, 2 hours, 4 hours, 6 hours, 12 hours, 24 hours, 36 hours, 48 hours, 5 days, 1 week, 2 weeks, 1 month or more after), or concomitantly with the administration of one or more therapeutic agents other than an active vitamin D compound or a mimic thereof.

In accordance with the present invention, one or more active vitamin D compounds or mimics thereof may be advantageously utilized in combination with one or more anti-angiogenic factors (e.g., angiostatin or endostatin), one or more TNF-α antagonists (e.g., anti-TNF-α antibody), one or more integrin αᵥβ₃ antagonists, one or more anti-inflammatory agents, one or more immunomodulatory agents and/or one or more dermatological agents. Such combinational use may reduce adverse side effects associated with the administration of both the active vitamin D compound or a mimic thereof and the other therapeutic agent.

In a specific embodiment, the administration of one or more active vitamin D compounds or mimics thereof reduces the dosage and/or frequency of administration of one or more dosages of known therapeutic agents for the treatment or amelioration of a particular immune-mediated disorder. For example, the normally recommended starting dose for injection of tacrolimus (FK 506, an immunosuppression agent), 0.03 to 0.05 mg/kg per day as a continuous infusion, for the treatment of solid organ allograft may be reduced by the administration of an active vitamin D compound or a mimic thereof. Further, the recommended initial oral dose of tacrolimus for adult kidney transplant patients (0.2 mg/kg per day), adult liver transplant patients (0.1 to 0.15 mg/kg per day), and pediatric liver transplant patients (0.15 to 0.2 mg/kg per day in two divided doses 12 hours apart) may be reduced by the administration of an active vitamin D compound or a mimic thereof. In a preferred embodiment, an active vitamin D compound or a mimic thereof is administered once every three or more days during the treatment with tacrolimus, which can be administered at a lower dosage or less frequently without compromising its therapeutic effects.

Examples of therapeutic agents used to treat or ameliorate rheumatoid arthritis include, but are not limited to, Remicade, corticosteroids, tacrolimus, bisphosphonates, NSAEDs (e.g., ibuprofen, fenprofen, indomethacin, and naproxen), anti-malarial drugs (e.g., hydroxychloroquine and sulfasalazine), Anakinra, azathioprine, Enbrel, Celebrex, and cyclophosphamide. Examples of therapeutic agents used to treat or ameliorate Crohn's disease include, but are not limited to, sulfasalazine (Azulfidine), aminosalicylates, steroids (e.g., prednisone), and infliximab. Examples of therapeutic agents used to treat or ameliorate systemic lupus erythematosus include, but are not limited to, NSAIDs, antimalarial drugs (e.g., hydroxychloroquine), corticosteroids, glucocorticoids (e.g., triamcinolone), methotrexate, and azathioprine. Examples of therapeutic agents used to treat or ameliorate asthma include, but are not limited to, corticosteroids (Azmacort, Vanceril, AeroBid, Flovent, prednisone, methylprednisone, and hydrocortisone), leukotriene inhibitors, aminophylline and theophylline. Examples of therapeutic agents used to treat or ameliorate autoimmune hepatitis include, but are not limited to, corticosteroids (e.g., prednisone), azathiopurine and mercaptopurine. Examples of therapeutic agents used to treat, ameliorate, or prevent transplant rejection include, but are not limited to, azathioprine, cyclosporine, mycophenolate mofetil, rapamune, corticosteroids, and OKT2 monoclonal antibodies. One example of a therapeutic agent used to treat or ameliorate multiple sclerosis is IFN-1a (Avonex).

Examples of therapeutic agents used to treat or ameliorate bullous systemic lupus include, but are not limited to, dapsone, corticosteroids (e.g., prednisone and triamcinolone), and methotrexate. Examples of therapeutic agents used to treat or ameliorate scleroderma include, but are not limited to, prednisone, azathioprine, methotrexate, cyclophosphamide, and penicillamine. Examples of therapeutic agents used to treat or ameliorate pyoderma gangrenosum include, but are not limited to, prednisone, azathioprine, cyclophosphamide, chlorambucil, tacrolimus, immune globulins, and thalidomide. Examples of therapeutic agents used to treat or ameliorate alopecia areata include, but are not limited to, cyclosporine, methoxsalen, anthralin, clobetiasol propionate, prednisone, triamcinolone, betamethasone, and minoxidil. Examples of therapeutic agents used to treat or ameliorate vitiligo include, but are not limited to, triamcinolone, hydrocortisone, prednisone, methoxsalen, and trioxsalen. Examples of therapeutic agents used to treat or ameliorate contact dermatitis include, but are not limited to, clobetasol, hydrocortisone, prednisone, triamcinalone, hydroxyzine, doxepin, and disulfiran. Examples of known treatments for psoriasis include, but are not limited to, hydroxyurea, methotrexate, cyclosporin, acitretin, ultraviolet B radiation phototherapy, photochemotherapy, topical corticosteroids (e.g., diflorasone diacetate, clobetasol propionate, halobetasol propionate, betamethasone dipropionate, fluocinonide, halcinonide, desoximetasone, triamcinolone acetonide, fluticasone propionate, flucinolone acetonide, flurandrenolide, mometasone furoate, betamethasone, aclometasome dipropionate, desonide, and hydrocortisone), dithranol (anthralin), coal tar, salicylic acid, topical retinoids (e.g., tazarotene), macrolide antibiotics (e.g., tacrolimus), anti-CD3 monoclonal antibodies, anti-CD4 monoclonal antibodies, anti-CD11a monoclonal antibodies, anti-IL-2Rα monoclonal antibodies, anti-ICAM 1 antibodies, anti-LFA1 antibodies, anti-CD80 monoclonal antibodies, CTLA4Ig, and emollients. For reviews of treatments for psoriasis see, e.g., Ashcroft. et al., J. of Clin. Pharm. and Therap. 25:1-10 (2000); Karasek, Cutis 64:319-322 (1999); Drew, Primary Care 27:385-406 (2000); Lebwohl, Dermatologic Clinics 18:13-19 (2000); and Peters et al., Am. J. Health-Sys. Pharm. 57:645-659 (2000). In a specific embodiment, one or more active vitamin D compounds or mimics thereof are administered to a human to treat or ameliorate psoriasis prior to (e.g., 0.5 hours, 1 hour, 2 hours, 4 hours, 6 hours, 12 hours, 24 hours, 36 hours, 48 hours, 5 days, 1 week, 2 weeks, 1 month or more before), subsequent to (e.g., 0.5 hours, 1 hour, 2 hours, 4 hours, 6 hours, 12 hours, 24 hours, 36 hours, 48 hours, 5 days, 1 week, 2 weeks, 1 month or more after), or concomitantly with the administration of hydroxyurea, methotrexate, cyclosporin, acitretin, ultraviolet B radiation phototherapy, photochemotherapy, one or more topical corticosteroids, dithranol, coal tar, salicylic acid, IL-10, one or more topical retinoids, one or more macrolide antibiotics, one or more anti-CD3 monoclonal antibodies, one or more anti-CD4 monoclonal antibodies, one or more anti-CD11a monoclonal antibodies, one or more anti-IL-2Rα monoclonal antibodies, one or more anti-ICAM 1 antibodies, one or more anti-LFA1 antibodies, one or more anti-CD80 monoclonal antibodies, CTLA4Ig, or one or more emollients to said human. In another embodiment, one or more active vitamin D compounds are administered to an animal, preferably a human, with psoriasis prior to (e.g., 0.5 hours, 1 hour, 2 hours, 4 hours, 6 hours, 12 hours, 24 hours, 36 hours, 48 hours, 5 days, 1 week, 2 weeks, 1 month or more before), subsequent to (e.g., 0.5 hours, 1 hour, 2 hours, 4 hours, 6 hours, 12 hours, 24 hours, 36 hours, 48 hours, 5 days, 1 week, 2 weeks, 1 month or more after), or concomitantly with the administration of Xanelim (Genentech/Xoma), Enbril (Immunex, Inc.), Remicade (J&J/Centocor), ABX-IL-8 (Abgenix), IDEC-114 (IDEC Pharmaceuticals, Inc.), Novim (PDL, Inc.), Zenapax (PDL, Inc.), and/or Amevive (Biogen, Inc.).

The active vitamin D compound or a mimic thereof and one or more therapeutic agents of the combination therapies of the present invention can be administered concomitantly or sequentially to an animal. The active vitamin D compound or a mimic thereof and one or more therapeutic agents of the combination therapies of the present invention can also be cyclically administered. Cycling therapy involves the administration of a first therapeutic agent for a period of time, followed by the administration of a second therapeutic agent for a period of time and repeating this sequential administration, i.e., the cycle, in order to reduce the development of resistance to one of the agents, to avoid or reduce the side effects of one of the agents, and/or to improve the efficacy of the treatment. The active vitamin D compound or a mimic thereof and one or more therapeutic agents of the combination therapies of the invention can be administered to a subject concurrently. The term “concurrently” is not limited to the administration of therapeutic agents at exactly the same time, but rather it is meant that a active vitamin D compound or a mimic thereof and the one or more therapeutic agents are administered to an animal in a sequence and within a time interval such that the active vitamin D compound or a mimic thereof can act together with the other agent(s) to provide an increased benefit than if they were administered otherwise. The active vitamin D compound or a mimic thereof and one or more therapeutic agents can be administered separately, in any appropriate form and by any suitable route. In preferred embodiments, the active vitamin D compound or a mimic thereof and one or more therapeutic agents are administered within the same patient visit. The active vitamin D compound or a mimic thereof and one or more therapeutic agents of the combination therapies can be administered to an animal in the same pharmaceutical composition. Alternatively, the active vitamin D compound or a mimic thereof and one or more therapeutic agents of the combination therapies can be administered concurrently to an animal in separate pharmaceutical compositions. The active vitamin D compound or a mimic thereof and one or more therapeutic agents may be administered to an animal by the same or different routes of administration.

Any period of treatment with the active vitamin D compound or a mimic thereof prior to the administration of the one or more therapeutic agents can be employed in the present invention. The exact period for treatment with the active vitamin D compound or a mimic thereof will vary depending upon the active vitamin D compound or a mimic thereof used, the immune-mediated disorder, the patient, and other related factors. The active vitamin D compound or a mimic thereof may be administered for as little as 12 hours and as much as 3 months prior to the administration of the one or more therapeutic agents. If the active vitamin D compound or a mimic thereof is administered daily, it may be administered for about 1 to about 10 days before administration of the one or more therapeutic agents. In certain embodiments, the methods of the invention comprise administering the active vitamin D compound or a mimic thereof daily for 1, 2, 3, 4, 5, 6, 7, 8, 9, or 10 days before administration of the one or more therapeutic agents. If the active vitamin D compound or a mimic thereof is administered in a pulsed-dose fashion, it may be administered at least one day before administration of the one or more therapeutic agents and for as long as 3 months before administration of the one or more therapeutic agents. In certain embodiments, the methods of the invention comprise administering the active vitamin D compound or a mimic thereof once every 3, 4, 5, 6, 7, 8, 9, or 10 days for a period of 3 days to 60 days before administration of the one or more therapeutic agents.

The administration of the active vitamin D compound or a mimic thereof, in either a daily or pulsed-dose manner, may be continued concurrently with the administration of the one or more therapeutic agents.

Additionally, the administration of the active vitamin D compound or a mimic thereof may be continued beyond the administration of the one or more therapeutic agents.

In certain embodiments of the invention, the method of administering an active vitamin D compound or a mimic thereof in combination with one or more therapeutic agents may be repeated at least once. The method my be repeated as many times as necessary to achieve or maintain a therapeutic response, e.g., from one to about ten times. With each repetition of the method the active vitamin D compound or a mimic thereof and the one or more therapeutic agents may be the same or different from that used in the previous repetition. Additionally, the time period of administration of the active vitamin D compound or a mimic thereof and the manner in which it is administered (i.e., daily or pulsed-dose) can vary from repetition to repetition.

In certain embodiments, a therapeutic or pharmaceutical composition of the invention is administered prior to or after the presence of the symptoms or diagnosis of disease. In a specific embodiment, the combinatorial therapies of the invention do not induce relative to single agent therapies or other known combination therapies one or more of the following unwanted or adverse effects: vital sign abnormalities (fever, tachycardia, bradycardia, hypertension, hypotension), hypercalcemia, hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, vasodilatation, an increased risk of opportunistic infection, and an increased risk of developing certain types of cancer.

Animals which may be treated according to the present invention include all animals which may benefit from administration of the compounds of the present invention. Such animals include humans, pets such as dogs and cats, and veterinary animals such as cows, pigs, sheep, goats and the like.

The following examples are illustrative, but not limiting, of the methods of the present invention. Other suitable modifications and adaptations of the variety of conditions and parameters normally encountered in medical treatment and pharmaceutical science and which are obvious to those skilled in the art are within the spirit and scope of the invention.

### EXAMPLE 1

### Preparation of Semi-solid Calcitriol Formulations

Five semi-solid calcitriol formulations (SS1-SS5) were prepared containing the ingredients listed in Table 1. The final formulation contains 0.208 mg calcitriol per gram of semi-solid formulation.

TABLE 1
Composition of Semi-Solid Calcitriol Formulation

| Ingredients | SS1 | SS2 | SS3 | SS4 | SS5 |
|---|---|---|---|---|---|
| Calcitriol | 0.0208 | 0.0208 | 0.0208 | 0.0208 | 0.0208 |
| Miglyol 812 | 80.0 | 0 | 65.0 | 0 | 79.0 |
| Captex 200 | 0 | 82.0 | 0 | 60.0 | 0 |
| Labrafac CC | 0 | 0 | 0 | 0 | 12.0 |
| Vitamin-E TPGS | 20.0 | 18.0 | 5.0 | 5.0 | 9.0 |
| Labrifil M | 0 | 0 | 0 | 0 | 0 |
| Gelucire 44/14 | 0 | 0 | 30.0 | 35.0 | 0 |
| BHT | 0.05 | 0.05 | 0.05 | 0.05 | 0.05 |
| BHA | 0.05 | 0.05 | 0.05 | 0.05 | 0.05 |

Amounts shown are in grams.

1. Preparation of Vehicles

One hundred gram quantities of the five semi-solid calcitriol formulations (SS1-SS5) listed in Table 1 were prepared as follows.

The listed ingredients, except for calcitriol, were combined in a suitable glass container and mixed until homogenous. Vitamin E TPGS and GELUCIRE 44/14 were heated and homogenized at 60° C. prior to weighing and adding into the formulation.

2. Preparation of Active Formulations

The semi-solid vehicles were heated and homogenized at ≦60° C. Under subdued light, 12±1 mg of calcitriol was weighed out into separate glass bottles with screw caps, one bottle for each formulation. (Calcitriol is light sensitive; subdued light/red light should be used when working with calcitriol/calcitriol formulations.) The exact weight was recorded to 0.1 mg. The caps were then placed on the bottles as soon as the calcitriol had been placed into the bottles. Next, the amount of each vehicle required to bring the concentration to 0.208 mg/g was calculated using the following formula:
C_{w}/0.208=required weight of vehicle
Where C_{w}=weight of calcitriol, in mg, and
0.1208=final concentration of calcitriol (mg/g).

Finally, the appropriate amount of each vehicle was added to the respective bottle containing the calcitriol. The formulations were heated (≦60° C.) while being mixed to dissolve the calcitriol.

### EXAMPLE 2

### Preparation of Additional Formulations

Following the method of Example 1, twelve different formulations for calcitriol were prepared containing the ingredients listed in Table 2.

TABLE 2
Composition Formulations

| Ingredients | 1 | 2 | 3 | 4 | 5 | 6 | 7 | 8 | 9 | 10 | 11 | 12 |
|---|---|---|---|---|---|---|---|---|---|---|---|---|
| Miglyol | 95 | 65 | 90 | 85 | 80 | 95 | 65 | 90 | 85 | 80 | 50 | 0 |
| 812N | | | | | | | | | | | | |
| Vitamin | 5 | 5 | 10 | 5 | 10 | 5 | 5 | 10 | 5 | 10 | 50 | 50 |
| E TPGS | | | | | | | | | | | | |
| PEG | 0 | 30 | 0 | 10 | 10 | 0 | 30 | 0 | 10 | 10 | 0 | 50 |
| 4000 | | | | | | | | | | | | |
| BHA | 0.05 | 0.05 | 0.05 | 0.05 | 0.05 | 0.35 | 0.35 | 0.35 | 0.35 | 0.35 | 0.35 | 0.35 |
| BHT | 0.05 | 0.05 | 0.05 | 0.05 | 0.05 | 0.35 | 0.35 | 0.35 | 0.35 | 0.35 | 0.35 | 0.35 |

Amounts shown are percentages.

### EXAMPLE 3

### Stable Unit Dose Formulations

Formulations of calcitriol were prepared to yield the compositions in Table 3. The Vitamin E TPGS was warmed to approximately 50° C. and mixed in the appropriate ratio with MIGLYOL 812. BHA and BHT were added to each formulation to achieve 0.35% w/w of each in the final preparations.

TABLE 3
Calcitriol formulations

| | MIGLYOL | Vitamin E TPGS |
|---|---|---|
| Formulation # | (% wt/wt) | (% wt/wt) |
| 1 | 100 | 0 |
| 2 | 95 | 5 |
| 3 | 90 | 10 |
| 4 | 50 | 50 |

After formulation preparation, Formulations 2-4 were heated to approximately 50° C. and mixed with calcitriol to produce 0.1 μg calcitriol/mg total formulation. The formulations contained calcitriol were then added (˜250 μL) to a 25 mL volumetric flask and deionized water was added to the 25 mL mark. The solutions were then vortexed and the absorbance of each formulation was measured at 400 nm immediately after mixing (initial) and up to 10 min after mixing. As shown in Table 4, all three formulations produced an opalescent solution upon mixing with water. Formulation 4 appeared to form a stable suspension with no observable change in absorbance at 400 nm after 10 min.

TABLE 4
Absorption of formulations suspended in water

Absorbance at 400 nm

| Formulation # | Initial | 10 min |
|---|---|---|
| 2 | 0.7705 | 0.6010 |
| 3 | 1.2312 | 1.1560 |
| 4 | 3.1265 | 3.1265 |

To further assess the formulations of calcitriol, a solubility study was conducted to evaluate the amount of calcitriol soluble in each formulation. Calcitriol concentrations from 0.1 to 0.6 μg calcitriol/mg formulation were prepared by heating the formulations to 50° C. followed by addition of the appropriate mass of calcitriol. The formulations were then allowed to cool to room temperature and the presence of undissolved calcitriol was determined by a light microscope with and without polarizing light. For each formulation, calcitriol was soluble at the highest concentration tested, 0.6 μg calcitriol/mg formulation.

A 45 μg calcitriol dose is currently being used in Phase 2 human clinical trials. To develop a capsule with this dosage each formulation was prepared with 0.2 μg calcitriol/mg formulation and 0.35% w/w of both BHA and BHT. The bulk formulation mixtures were filled into Size 3 hard gelatin capsules at a mass of 225 mg (45 μg calcitriol). The capsules were then analyzed for stability at 5° C., 25° C./60% relative humidity (RH), 30° C./65% RH, and 40° C./75% RH. At the appropriate time points, the stability samples were analyzed for content of intact calcitriol and dissolution of the capsules. The calcitriol content of the capsules was determined by dissolving three opened capsules in 5 mL of methanol and held at 5° C. prior to analysis. The dissolved samples were then analyzed by reversed phase HPLC. A Phemonex Hypersil BDS C18 column at 30° C. was used with a gradient of acetonitrile from 55% acetonitrile in water to 95% acetonitrile at a flow rate of 1.0 mL/min during elution. Peaks were detected at 265 nm and a 25 μL sample was injected for each run. The peak area of the sample was compared to a reference standard to calculate the calcitriol content as reported in Table 5. The dissolution test was performed by placing one capsule in each of six low volume dissolution containers with 50 mL of deionized water containing 0.5% sodium dodecyl sulfate. Samples were taken at 30, 60 and 90 min after mixing at 75 rpm and 37° C. Calcitriol content of the samples was determined by injection of 100 μL samples onto a Betasil C18 column operated at 1 mL/min with a mobile phase of 50:40:10 acetonitrile:water:tetrahydrofuran at 30° C. (peak detection at 265 nm). The mean value from the 90 min dissolution test results of the six capsules was reported (Table 6).

TABLE 5
Chemical stability of calcitriol formulation in hard gelatin capsules
(225 mg total mass filled per capsule, 45 μg calcitriol)

| Storage | Time | Assay^{a} (%) | | | |
|---|---|---|---|---|---|
| Condition | (mos) | Form. 1 | Form. 2 | Form 3 | Form 4 |
| N/A | 0 | 100.1 | 98.8 | 99.1 | 100.3 |
| 5° C. | 1.0 | 99.4 | 98.9 | 98.9 | 104.3 |
| 25° C./60% RH | 0.5 | 99.4 | 97.7 | 97.8 | 102.3 |
| | 1.0 | 97.1 | 95.8 | 97.8 | 100.3 |
| | 3.0 | 95.2 | 93.6 | 96.8 | 97.9 |
| 30° C./65% RH | 0.5 | 98.7 | 97.7 | 96.8 | 100.7 |
| | 1.0 | 95.8 | 96.3 | 97.3 | 100.4 |
| | 3.0 | 94.2 | 93.6 | 95.5 | 93.4 |
| 40° C./75% RH | 0.5 | 96.4 | 96.7 | 98.2 | 97.1 |
| | 1.0 | 96.1 | 98.6 | 98.5 | 99.3 |
| | 3.0 | 92.3 | 92.4 | 93.0 | 96.4 |

^{a}Assay results indicate % of calcitriol relative to expected value based upon 45 μg content per capsule. Values include pre-calcitriol which is an active isomer of calcitriol.

TABLE 6
Physical Stability of Calcitriol Formulation in Hard Gelatin
Capsules (225 mg total mass filled per capsule, 45 μg calcitriol)

| Storage | Time | Dissolution^{a} (%) | | | |
|---|---|---|---|---|---|
| Condition | (mos) | Form. 1 | Form. 2 | Form 3 | Form 4 |
| N/A | 0 | 70.5 | 93.9 | 92.1 | 100.1 |
| 5° C. | 1.0 | 71.0 | 92.3 | 96.0 | 100.4 |
| 25° C./60% RH | 0.5 | 65.0 | 89.0 | 90.1 | 98.3 |
| | 1.0 | 66.1 | 90.8 | 94.5 | 96.2 |
| | 3.0 | 64.3 | 85.5 | 90.0 | 91.4 |
| 30° C./65% RH | 0.5 | 62.1 | 88.8 | 91.5 | 97.9 |
| | 1.0 | 65.1 | 89.4 | 95.5 | 98.1 |
| | 3.0 | 57.7 | 86.4 | 89.5 | 88.8 |
| 40° C./75% RH | 0.5 | 91.9 | 90.2 | 92.9 | 93.1 |
| | 1.0 | 63.4 | 93.8 | 94.5 | 95.2 |
| | 3.0 | 59.3 | 83.6 | 87.4 | 91.1 |

^{a}Dissolution of capsules was performed as described and the % calcitriol is calculated based upon a standard and the expected content of 45 μg calcitriol per capsule. The active isomer, pre-calcitriol, is not included in the calculation of % calcitriol dissolved. Values reported are from the 90 min sample.

The chemical stability results indicated that decreasing the MIGLYOL 812 content with a concomitant increase in Vitamin E TPGS content provided enhanced recovery of intact calcitriol as noted in Table 5. Formulation 4 (50:50 MIGLYOL 812/Vitamin E TPGS) was the most chemically stable formulation with only minor decreases in recovery of intact calcitriol after 3 months at 25° C./60% RH, enabling room temperature storage.

The physical stability of the formulations was assessed by the dissolution behavior of the capsules after storage at each stability condition.

As with the chemical stability, decreasing the MIGLYOL 812 content and increasing the Vitamin E TPGS content improved the dissolution properties of the formulation (Table 6). Formulation 4 (50:50 MIGLYOL 812/Vitamin E TPGS) had the best dissolution properties with suitable stability for room temperature storage.

Having now fully described the invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method for treating or ameliorating an immune-mediated disorder in an animal comprising administering to the animal a therapeutically effective amount of an active vitamin D compound or a mimic thereof in a pulsed-dose fashion no more frequently than once in three days.

2. The method of claim 1, wherein said immune-mediated disorder is an autoimmune disorder or an inflammatory disorder.

3. A method for treating, ameliorating, or preventing transplant rejection in an animal comprising administering to the animal a therapeutically effective amount of an active vitamin D compound or a mimic thereof in a pulsed-dose fashion no more frequently than once in three days.

4. The method of claims 1 or 3, further comprising administering one or more therapeutic agents.

5. The method of claims 1 or 3, wherein said active vitamin D compound or a mimic thereof is calcitriol.

6. The method of claims 1 or 3, wherein said active vitamin D compound or a mimic thereof is 25-OH vitamin D₃.

7. The method of claims 1 or 3, wherein said active vitamin D compound or a mimic thereof has a reduced hypercalcemic effect.

8. The method of claim 7, wherein said active vitamin D compound or a mimic thereof is selected from the group consisting of EB 1089, Ro23-7553, and Ro24-5531.

9. The method of claims 1 or 3, wherein said pulsed-dose is administered no more frequently than once in four days.

10. The method of claim 9, wherein said pulsed-dose is administered no more frequently than once a week.

11. The method of claims 1 or 3, wherein said active vitamin D compound or a mimic thereof is administered at a dose of about 15 μg to about 1 mg.

12. The method of claim 11, wherein said active vitamin D compound or a mimic thereof is administered at a dose of about 15 μg to about 90 μg.

13. The method of claim 12, wherein said active vitamin D compound or a mimic thereof is administered at a dose of about 25 μg to about 75 μg.

14. The method of claim 13, wherein said active vitamin D compound or a mimic thereof is administered at a dose of about 30 μg to about 60 μg.

15. The method of claim 14, wherein said active vitamin D compound or a mimic thereof is administered at a dose of about 45 μg.

16. The method of claims 1 or 3, wherein said active vitamin D compound or a mimic thereof is administered at a dose sufficient to obtain a peak plasma concentration of the active vitamin D compound of at least 0.5 nM.

17. The method of claims 1 or 3, wherein said active vitamin D compound or a mimic thereof is administered orally, intravenously, parenterally, rectally, topically, nasally or transdermally.

18. The method of claim 17, wherein said active vitamin D compound or a mimic thereof is administered orally or intravenously.

19. The method of claims 1 or 3, further comprising reducing the level of calcium in the blood of the animal.

20. The method of claim 19, wherein said reducing comprises eating a reduced calcium diet, trapping calcium with an adsorbent, absorbent, ligand, chelate, or other calcium binding moiety that cannot be transported into the blood through the small intestine, administering a bisphosphonate, increasing hydration and salt intake, or diuretic therapy.

21. The method of claim 4, wherein said one or more therapeutic agents are selected from the group consisting of an immunomodulatory agent, an anti-angiogenic agent, an anti-inflammatory agent, a dermatological agent, and any combination thereof.

22. The method of claim 4, wherein said active vitamin D compound or a mimic thereof is administered at least 12 hours prior to the administration of said one or more therapeutic agents.

23. The method of claim 22, wherein said active vitamin D compound or a mimic thereof is administered in a pulsed-dose fashion for 1 day to about 3 months prior to the administration of said one or more therapeutic agents.

24. The method of claim 4, wherein said active vitamin D compound or a mimic thereof is administered concurrently with the administration of said one or more therapeutic agents.

25. The method of claim 4, wherein the administration of said active vitamin D compound or a mimic thereof is continued beyond the administration of said one or more therapeutic agents.

26. The method of claim 4, wherein the active vitamin D compound or a mimic thereof is administered after the administration of said one or more therapeutic agents.

27. The method of claim 4, wherein the method is repeated at least once.

28. The method of claim 27, wherein the method is repeated one time to about 10 times.

29. The method of claim 27, wherein said active vitamin D compound or a mimic thereof may be the same or different in each repetition and said one or more therapeutic agents may be the same or different in each repetition.

30. The method of claim 27, wherein the time period of administration of said active vitamin D compound or a mimic thereof may be the same or different in each repetition.

31. The method of claim 1, wherein said active vitamin D compound or a mimic thereof is administered as a unit dosage form comprising about 10 μg to about 75 μg of calcitriol, about 50% MIGLYOL 812 and about 50% tocopherol PEG-1000 succinate (vitamin E TPGS).

32. The method of claim 31, wherein said unit dosage form comprises about 45 μg of calcitriol.

33. The method of claim 31, wherein said unit dosage form further comprises at least one additive selected from the group consisting of an antioxidant, a bufferant, an antifoaming agent, a detackifier, a preservative, a chelating agent, a viscomodulator, a tonicifier, a flavorant, a colorant, an odorant, an opacifier, a suspending agent, a binder, a filler, a plasticizer, a thickening agent, a lubricant, and mixtures thereof.

34. The method of claim 33, wherein one of said additives is an antioxidant.

35. The method of claim 34, wherein said antioxidant is selected from the group consisting of butylated hydroxyanisole (BHA) and butylated hydroxytoluene (BHT).

36. The method of claim 35, wherein said unit dosage form comprises BHA and BHT.

37. The method of claim 36, wherein said unit dosage form comprises about 50% MIGLYOL 812, about 50% vitamin E TPGS, about 0.05% to about 0.35% BHA, and about 0.05% to about 0.35% BHT.

38. The method of claim 37, wherein said unit dosage form comprises about 50% MIGLYOL 812, about 50% vitamin E TPGS, about 0.35% BHA, and about 0.10% BHT.

39. The method of claim 31, wherein said unit dosage form is a capsule.

40. The method of claim 39, wherein said capsule is a gelatin capsule.

41. The method of claim 39, wherein the total volume of ingredients in said capsule is 10-1000 μl.

42. The method of claim 31, wherein said unit dosage form comprises about 45 μg of calcitriol, about 50% MIGLYOL 812, about 50% vitamin E TPGS, BHA, and BHT.

43. The method of claim 31, wherein said unit dosage form comprises about 45 μg of calcitriol, about 50% MIGLYOL 812, about 50% vitamin E TPGS, about 0.35% BHA, and about 0.10% BHT.
